(19) United States
(12) Patent Application Publication
(10) Pub. No.: US 2018/0301220 A1
(43) Pub. Date: Oct. 18, 2018

(54) Wearable Sensor Packages and Health Score Analytics for Athletic Performance and Training

(71) Applicant: Steven I Rothman, Sarasota, FL (US)

(72) Inventor: Steven I. Rothman, Sarasota, FL (US)

(21) Appl. No.: 16/013,904

(22) Filed: Jun. 20, 2018

Related U.S. Application Data
(63) Continuation of application No. 15/908,803, filed on Feb. 28, 2018.
(60) Provisional application No. 62/465,039, filed on Feb. 28, 2017.

(51) Int. Cl. G16H 40/67; A61B 5/00; A61B 5/145

(52) U.S. Cl. CPC G16H 40/67; A61B 5/0006; A61B 5/681; A61B 5/02416; A61B 5/14542; A61B 5/0022

(57) ABSTRACT

Technologies are described for wearable sensor devices and associated analytic systems. Patient data may be collected from the wearable sensors, instruments, caregivers, or from other such devices. The wearable sensor devices may be calibrated according to the collected patient data. The collected data may be calibrated, normalized, and analyzed to generate and track health scores associated the patient. The wearable sensor devices may be leveraged to refine athletic performance, training, and strategy. The wearable sensor devices may be leveraged to predict and detect adverse health events.

### CROSS-REFERENCE TO RELATED APPLICATION

This application is a continuation of U.S. patent application Ser. No. 15/908,803, filed on Feb. 28, 2018, entitled “Intelligent Wearable Sensors for Adaptive Health Score Analytics,” which claims the benefit of U.S. Provisional Patent Application No. 62/465,039, filed on Feb. 28, 2017, entitled “Wearable Sensor Devices for Adaptive Health Score Tracking,” both of which are expressly incorporated herein by reference in their entirety.

### BACKGROUND

Patients in hospitals are almost constantly monitored and attended to by nursing staff and technicians. Physicians and specialists also generally see the patients throughout the day. The various professionals interacting with such patients have numerous opportunities to observe and evaluate the progress of each patient. There are numerous challenges to providing such close health monitoring and evaluation to individuals in their homes, in assisted living, or even in nursing homes or hospice care. Inadequate monitoring of patients often results in unobserved, and thus uncorrected, declines in the condition of the patient. This may often result in negative health outcomes, hospital (re)admissions, delayed recovery, complications, pain, discomfort, suffering, increased costs, and possibly death.

There is a need in the art for automated systems to monitor and evaluate the general overall health trends of a patient residing at home, in assisted living, or in a nursing facility. There is further need for such systems to leverage wearable sensor packages to collect patient metrics in a frequent and non-invasive manner to be incorporated into a trackable health score for evaluating progress, generating alerts, and establishing recommendations for transitions in the levels of monitoring and care.

### SUMMARY

The methods and systems described herein leverage wearable physiological sensors and a network of supporting technology to provide adaptive complimentary self-assessment and automated health scoring. Monitoring and tracking the health and wellbeing of patients in their homes, assisted living, nursing homes, hospice care, rehabilitation centers, and other healthcare facilities can be vital to identifying changes in health indicators. Certain changes in health indicators may indicate progress and recovery while certain other changes may precede catastrophic health events. Such monitoring and tracking may be particularly meaningful in the days and weeks immediately following discharge from a hospital or other care facility to ensure effective transition, reduce bad outcomes, and avoid readmissions. Such monitoring and tracing may also be useful near the transition points between levels of patient care such as prior and during the transition from patient home to nursing home or similar transition to hospice care.

Patient data may be collected from wearable sensors, instruments, caregivers, or from other such devices. The data may be calibrated, normalized, and analyzed to generate and track health scores for the patient. Data may also be derived from self-assessed patient query responses. The self-assessed patient query responses may be made in response to patient queries. The patient queries may be presented to the patient in conjunction with their wearable sensor systems, or through a computerized patient device (such as a tablet computer or smartphone). Patient queries may also be administered using automated voice calls, telephone calls, or any other electronic device.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

FIG. 1 illustrates three views of a wearable sensor device in accordance with one or more embodiments presented herein.

FIG. 2 is a block diagram illustrating a health score system in accordance with one or more embodiments presented herein.

FIG. 3 illustrates two views of an extended remote patch in accordance with one or more embodiments presented herein.

FIG. 4 is a hierarchical diagram illustrating six levels of wearable sensor monitoring functionality in accordance with one or more embodiments presented herein.

FIG. 5 is a chart showing a health score graph in accordance with one or more embodiments presented herein.

FIG. 6 is a graph of an excess risk curve where an outcome measurement is a function of heart rate in accordance with one or more embodiments presented herein.

FIG. 7 is a graph of an excess risk curve where an outcome measurement is a function of creatinine in accordance with one or more embodiments presented herein.

FIG. 8 is a block flow diagram depicting a method for applying wearable sensor data to adaptive health score tracking in accordance with one or more embodiments presented herein.

FIG. 9 is a block flow diagram depicting a method for applying wearable sensor packages and health score analytics to athletic performance and training in accordance with one or more embodiments presented herein

FIG. 10 is a block flow diagram depicting a method 1000 for applying wearable sensor packages and health score analytics to the prediction and detection of adverse health events in accordance with one or more embodiments presented herein.

FIG. 11 is a block flow diagram depicting a method for assessing risk associated with at least one type of patient data due to deviation from normative values in accordance with one or more embodiments presented herein.

FIG. 12 is a block flow diagram depicting a method for determining an overall risk associated with the current health condition of a patient in accordance with one or more embodiments presented herein.

FIG. 13 is a block diagram depicting a computing machine and a module in accordance with one or more embodiments presented herein.

### DETAILED DESCRIPTION OF EXAMPLE EMBODIMENTS

### Overview

The functionality of various example embodiments will be explained in more detail in the following description to be read in conjunction with the figures illustrating system components and process flows. Turning now to the drawings, in which like numerals indicate like (but not necessarily identical) elements throughout the figures, example embodiments are described in detail.

### Example System Architectures

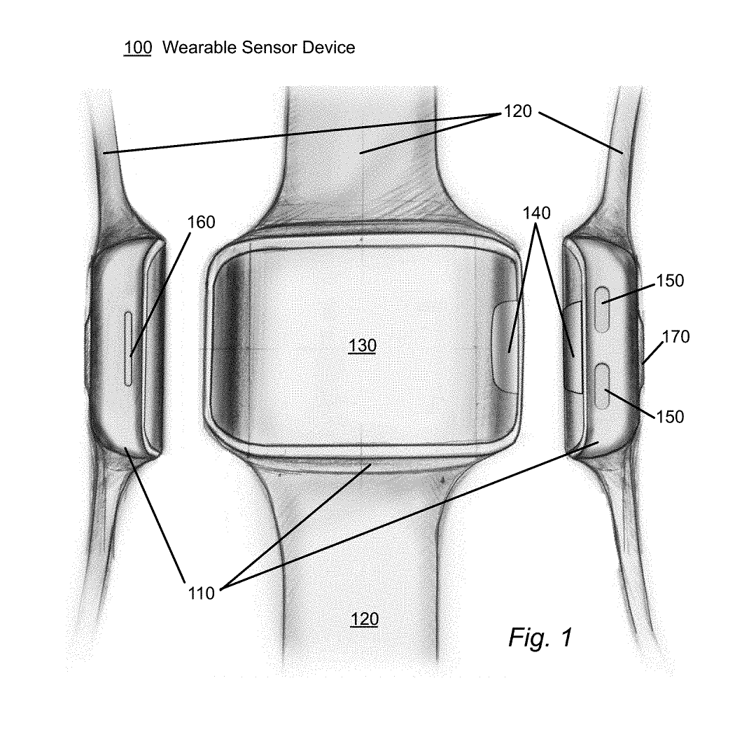

FIG. 1 illustrates three views of a wearable sensor device 100, in accordance with certain exemplary embodiments. The wearable sensor device 100 can collect and process patient data related to health and wellness. The wearable sensor device 100 may be worn on a human wrist (or other extremity) held in place by a strap 120. A primary enclosure 110 may contain electronics and sensors associated with the wearable sensor device 100. The primary enclosure 110 may include a display panel 130. A topside electrode 140 may be located on the primary enclosure 110 to support electrocardiograph (ECG or EKG) functionality. One or more buttons 150 may be located on the primary enclosure 110 to support user input. A SIM slot 160 may be located within the primary enclosure 110 to support mobile/cellular communication. Rear sensors 170 may be located on the primary enclosure 110 to support physiological sensors positioned proximate to the patient's skin.

The rear sensors 170 may include two-frequency Doppler radar, photoplethysmogram (PPG), electrocardiograph (ECG or EKG) electrodes, skin temperature sensors, skin conductivity/resistivity, and so forth.

The PPG sensor can support an optically obtained plethysmogram, which is a volumetric measurement of an organ. The PPG may be obtained using a pulse oximeter, which illuminates the skin and measures changes in light absorption. The pulse oximeter can monitor the perfusion of blood to the dermis and subcutaneous tissue of the skin. The PPG sensor may operate on multiple optical frequencies, so as three-frequencies, for example red, green, and infrared.

The EKG sensor can support multi-electrode operation by providing one electrode among the rear sensors 170 for contacting skin under the primary enclosure 110 and also a second topside electrode 140 for the user to contact with their opposite hand from the one having the wearable sensor device 100. The EKG sensor can intelligently detect various forms of cardiac arrhythmia, tachycardia, atrial fibrillation, and so forth.

The wearable sensor device 100 may include a speaker or buzzer to support audio/voice notifications and/or communications. The wearable sensor device 100 may include a microphone to support voice activation and communications. The microphone may also be used as input to audio signal analysis such as voice stress analysis. The wearable sensor device 100 may include a vibration motor or actuator to support silent user notifications.

The wearable sensor device 100 may include sensors for location, motion, and position such as a magnetometer, an accelerometer, and/or a gyroscope. These sensors may support nine or more degrees of freedom. The wearable sensor device 100 may include an altimeter, such as a barometer. The altimeter may provide a measurement accuracy of plus-or-minus three inches or better. The wearable sensor device 100 may support global positioning satellite (GPS) technology or similar radio frequency geolocation mechanisms.

The wearable sensor device 100 can support multiple modalities of wireless communications such as Wi-Fi, Bluetooth, BLE, LoRa, and mobile/cellular. The mobile cellular may support 2G, 3G, 4G, LTE, CDMA, or otherwise. One or more of the antennas associated with these communication modalities and/or the GPS receiver may be embedded within the strap 120. The wearable sensor device 100 can use one or more of the supported communication modalities to send all raw sensor data, digested/processed data, or any combination thereof to one or more supporting servers.

The wearable sensor device 100 can support NFC/RFID including a passive transponder. The transponder may be embedded within the strap 120.

The wearable sensor device 100 can support a long-life rechargeable battery and may be recharged via resting cradle or a cable. The wearable sensor device 100 can provide a rapid identity swapping functionality to support institutional operations where a patient's wearable sensor device 100 is swapped with a freshly charged one (without any loss of state or functionality) so that the original wearable sensor device 100 can be charged, cleaned or otherwise serviced.

The wearable sensor device 100 can provide various physiological metrics such as heart rate, heart rate variability, systolic blood pressure, diastolic blood pressure, oxygen saturation, temperature, and respiration rate. These metrics may contribute to computing and tracking a patient health score such as the Rothman Index. Self-assessment metrics from the patient may also contribute to the health score. These may be queried from the user via the wearable sensor device 100 or using a computer, mobile device, telephone call, or other mode of communication. Some or all of of the sensor and/or health score metrics may be available directly to user. Some or all of the metrics may be adaptively monitored and support generation of user-normalized alerts.

The wearable sensor device 100 can support “I have fallen and I can't get up” detection. This solution may not even require the user to press a button, but may automatically detect fall conditions. The wearable sensor device 100 can even support “I have not fallen but I can't get up” detection based upon sensing sustained inactivity while not sleeping. The wearable sensor device 100 can support trapped detection by sensing excessive time in attic, basement, bathroom, etc.

The wearable sensor device 100 can support emergency calls via mobile/cellular service to 911 or security or front desk or SSH dispatch service.

The wearable sensor device 100 can support “wander-guard” for memory care patients. The wearable sensor device 100 can activate or lock doors and/or alarms or alerts (RFID system), intercoms, call buttons (alert and/or talk to nursing station or front desk or security), GPS location, including remote location for wandering user or for lost sensor-band.

The wearable sensor device 100 can support diagnostic monitoring and alerts, such as arrhythmia, tachycardia, apnea, hypertension, sleep levels, and sleep duration.

The wearable sensor device 100 can support general fitness and wellness, such as step count, compass, distance, mapping, fitness test, activity level, food reminders, exercise assistance, and encouragement/coaching.

While many examples and illustration presented herein refer to the wearable sensor device 100 being worn on the wrist held in place by the strap 120, it should be appreciated that other modes of wearing the wearable sensor device 100 are within the scope of this technology. According to certain embodiments, the wearable sensor device 100 may be placed within an adhesive structure for attachment to the chest or other area of the patient's body. According to other embodiments, such as the adhesive structure example, the topside electrode 140 may be replaced by a second skin-facing electrode.

While many examples and illustration presented herein refer to the wearable sensor device 100 being worn by a human patient, it should be appreciated that the wearable sensor device 100 may be used in veterinary applications as well.

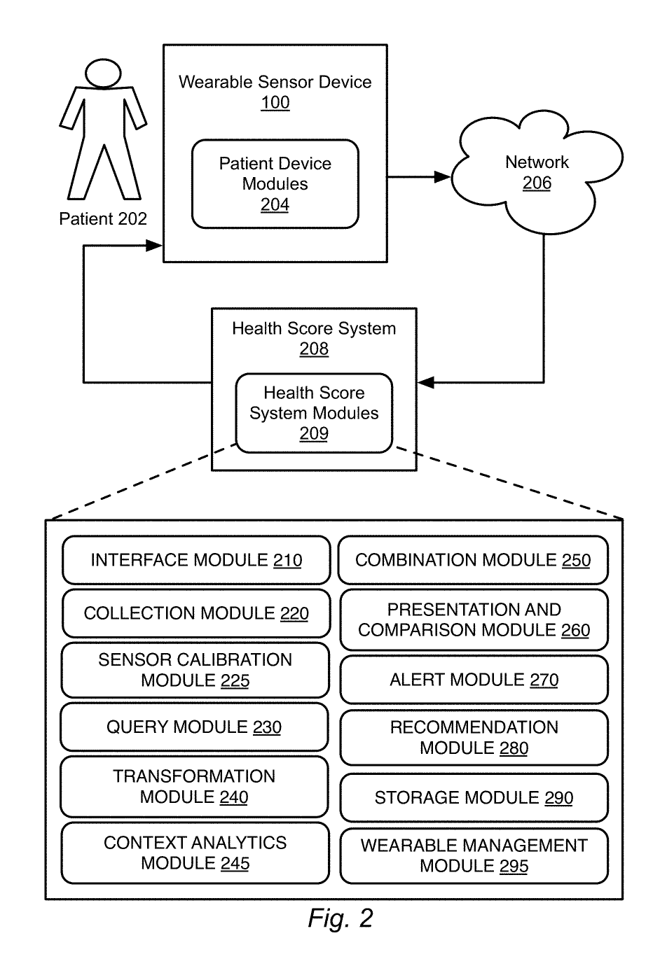

FIG. 2 is a block diagram illustrating a health score system 208, in accordance with certain exemplary embodiments. Patient data, such as signals from the wearable sensor device 100 and patient query responses, may be collected for processing by the health score system 208 executing one or more health score system modules 209. The health score system 208 may generate health score system outputs. The health score system outputs may include health scores, health score graphs, alerts, and/or recommendations. The health score system outputs may also include patient data collection specifications, which may then influence content, context, and/or timing of the patient queries.

Various recipients of the health score system outputs can include caregivers, caseworkers, family members, or friends/neighbors. The patient 202 may also be a direct recipient of the health score outputs. The recipients may use the provided health score system outputs to influence the patient data collection specification. Generation and refinement of the patient data collection specification by both the health score system 208 and one or more of the recipients may be referred to as a complimentary solution where machine intelligence and human intelligence compliment one another to address the challenges of patient monitoring and evaluation.

The patient 202 and their associated wearable sensor device 100 may be located in their home, independent living facility, retirement living facility, assisted living, hospice, nursing home, rehabilitation center, or some other such facility. The patient 202 may require or benefit from medical care or supervision in either an inpatient or outpatient setting. For example, the technology presented herein may support tracking the patient 202 after discharge from a facility or after a procedure. Such tracking may reduce incidences of readmission or poor outcomes. Self-assessment from the patient 202 may serve as partial input to the health score system 208. The health score system 208 may process the self-assessment inputs along with other inputs to generate health scores, health score graphs, alerts, and recommendations.

The patient 202 may interface with the complimentary self-assessment health scoring system via the wearable sensor device 100 along with any type of computing machine including a smart phone, tablet computer, set-top box, desktop computer, laptop, or so forth. The patient device modules 204 may include a real-time operating system (RTOS) and various associated modules to operate sensors and support interfacing with the patient 202 via the wearable sensor device 100. Patient queries may be presented to the patient 202 via a user interface associated with a computing machine or telephone to obtain patient query responses.

The wearable sensor device 100 may query the patient 202 with self-assessment questions (for example, using a speaker and a microphone) and may also directly measure physical qualities about the patient 202 (for example, temperature, heart rate, motion, and so forth).

According to certain embodiments, the wearable sensor device 100 in the form of a wristband, sensor band, smart watch, or other wearable device, may measure significantly more detailed information than traditional vital signs. For example, sampling numerous individual sensors within the wearable sensor device 100, such as sensors for pressure, vibration, or motion, may support the wearable sensor device 100 operating beyond detecting the pulse in simple beats per minute. Raw data from the numerous sensors may support analyzing details of a pulse waveform containing much more information than simple heart rate. Such a wearable sensor device 100 may be operable to detect arrhythmia, mean arterial pressure (MAP), and may provide something like an EKG trace from the patient. Detail enhancement in pulse measurement may support categorizing the waveforms to identify characteristics such as a weak pulse, thready pulse, or other types of cardiology information. Analysis of these sensor signals may include Fourier analysis, Hilbert analysis, correlations, and various other signal processing and signal analysis techniques. Such additional measured or computed data may be used as components in determining the health scores.

Even when the wearable sensor device 100, such as the wearable technology described above, do not report sufficient information to compute entire health scores on their own, the data that is supplied may be used to compute a portion of a health score or a partial health score. Such partial health scores may trigger the health score system 208 to collect or request additional patient data to compute one or more full health scores for assessment. Such a triggering signal may serve as an adaptive mechanism to the patient data collection specification. Perturbations in such a triggering signal may indicate that additional patient data would be helpful for further determination of heath score system outputs. Adaptive algorithms may also be applied to the sensitivity of the triggering signal.

The collected patient data may include patient query responses provided by the patient 202 in response to patent queries. The patient queries issued to the patient 202 can elicit a self-assessment from the patient 202 regarding their health or wellbeing. For example, the patient queries may interview the patient 202 about pain levels, activity levels, medication compliance, sleep quality, sleep duration, food intake, restroom visits, or other questions regarding general wellbeing. The patient queries may also question the patient 202 regarding self measurement of various physiological quantifies such as weight, temperature, blood pressure, blood sugar, pulse oximetry, and so forth. The patient queries may also present the patient 202 with assessment questions seeking to determine state of mind, mental acuity, attention, alertness, other psychological or other emotional qualities of the patient 202. The patient queries may also question the patient 202 regarding their need or desire for addition or reduced care interaction or their satisfaction level with current or recent care experiences.

The collected patient data may include secondary, subjective, or qualitative assessments of patient query responses. For example, audio signals provided as patient responses while self-assessment is gathered from a voice telephone system may be examined using voice stress analysis. Similarly, time taken for patients to respond, requests to repeat questions, delays, indications of confusion, or other such subjective patient characteristics may also be part of the collected patient data. This information, such as estimated stress or confusion may be considered self-assessment feedback and may used with other collected patient data, for example to compute one or more health scores. Voice stress analysis may also be performed from a microphone embedded within a wearable device (such as a smart watch). Voice stress analysis may be performed with respect to active voice signal collection, for example on voice responses to self-assessment queries. Voice stress analysis may be performed with respect to passive voice signal collection, for example on speech or other vocalizations of the patient at any time.

In addition to patient query responses, the collected patient data may include outputs from one or more patient sensors or instruments. Example instruments may include heart monitors, respiration monitors, oxygenation monitors, blood pressure monitors, and so forth. Similarly, example sensors may include motion sensors, proximity sensors, door switches, light sensors, temperature sensors, and so forth. The sensors or instruments may measure objective medical or lifestyle information about the patient 202. For example, information from the sensors may supplement, or corroborate, patient query responses to determine if the patient 202 is socializing, engaging in physical activities, getting around the house, eating, and/or visiting the restroom as expected. The collected patient data may include vital signs and various related measurements or evaluations of the patient.

The collected patient data may also be in the form of one or more caregiver reports. The caregiver reports may be provided by individuals such as home healthcare, nurses, technicians, physicians, caseworkers, friends, family, or neighbors. The various individuals interacting with the patient 202 may place their notes, observations, measurements, and so forth into the caregiver reports associated with the patient 202.

The various examples of patient data may be transmitted to the health score system 208. The health score system 208 may be configured to execute one or more health score system modules 209. The health score system 208 may process the patient data through various rules and algorithms to generate health score system outputs.

The health score system 208 can evaluate patient data to determine various metrics and trends in those metrics. One or more health scores may be derived from these metrics and trends within the metrics. The health scores may serve as indicators of the health status of the patient 202. For example, health scores that are generally improving over time after discharge can indicate recovery from a procedure or hospital stay. Alternatively, health scores that are suddenly declining may provide early indications of a complication such as infection or organ failure.

Health score system outputs may be generated by the health score system 208. The health score system outputs may include one or more health scores as well as health score graphs showing the progression of health scores over time or comparing various sets or standards of health scores. The health score system outputs may also include medical alerts. The alerts may be generated when one or more values form the patient data, alone or in combination, cross above or below specified thresholds. Alerts may also be generated when health scores cross above or below specified thresholds. In addition to crossing above or below specified thresholds, alerts may also be generated when certain values (or combinations of values) change too rapidly (e.g. a time derivative exceeds a certain threshold), or fail to change as expected. Alerts may also be generated when the patient 202 fails to respond to patient queries or appears to be doing so in an inappropriate or unexpected fashion.

The health score system outputs may also include various recommendations regarding future health care of the patient 202. The recommendations may include a suggested course of action for the patient 202 including suggested tests or procedures to be considered. The recommendations may also include suggestions to the patient 202 to another type, or category, of healthcare facility (for example, from a nursing home to hospice). The recommendations may also include evidentiary support for maintaining a certain level or type of care for a patient. The recommendations may also include suggested changes in types of medications, dosages of medications, dietary intake, physical activity levels, and so forth.

The health score system outputs may also include the patient data collection specification. The patient data collection specification can indicate the nature and frequency of patient queries. The health score system 208 can generate or update the patient data collection specification to adapt to a need for additional data points, or different type of data, from the patient 202. For example, a patient 202 who the health score system 208 has determined to potentially be in the early stage of a physiological decline may receive more frequent telephone calls, or computerized messages, to issue patient queries regarding the condition of the patient 202. The patient data collection specification may indicate the frequency of collecting various examples of patient data including those from patient sensors, instruments, and caregiver reports.

In general, the patient data collection specification can include specifications to commence, terminate, or modify patient data collection, which may also include information queries or instructions issued to the patient 202. The patient data collection specification may be derived from one or more adaptive intelligence algorithms in association with patient data. In addition to being generated by the health score system 208, the patient data collection specification may be complimented by feedback from one or more human recipients of the health score system outputs. The recipients may be caregivers, caseworkers, family, or friends/neighbors. The caregivers may be anyone practicing healthcare in a professional or non-professional capacity, such as physicians, nurses, technicians, family members and so forth.

The monitoring of health score system outputs by the recipients can significantly improve patient outcomes. The catastrophic deterioration of a patient's overall health is frequently preceded by documented deterioration of physiological parameters. Traditional rapid response teams in healthcare may be triggered by one parameter at a time, and that parameter often represents a significant change in a particular vital sign. For example, a significant change in blood pressure, or a significant change in skin color might trigger a call. In some cases, a general feeling that something is not right might lead to a call. Failure of clinical staff to respond to deterioration of respiratory or cerebral function and increase levels of medical intervention may often put patients at risk of cardio-respiratory arrest. In general, inappropriate action in response to observable abnormal physiological and biochemical variables might lead to avoidable death. The complimentary self-assessment health scoring system can significantly improve patient outcomes with regard to mortality, readmissions, and general patient satisfaction and wellbeing. These improvements may often be realized while also reducing costs.

During the course of care for a patient 202, they may see many caregivers such as technicians and nurses. Variations in these caregivers can make it extremely difficult to provide continuity of care. The patient 202 may have tens or hundreds of pages in their record, including progress reports, nursing evaluations, records of vital signs, test results, heart monitoring information, and so on. Even if every caregiver who saw the patient were fully aware of the material in this record, it may not be enough to allow for the best medical care because it is very difficult to detect trends in such voluminous data. The result of this arrangement has been to allow a number of patients in recovery, post-operation or procedure, to deteriorate to the point of medical crisis before addressing their problems. This causes a serious drain to the resources of the hospital, and unnecessary pain and suffering, even death. It is particularly bothersome because many of the conditions that lead to such crises can easily be avoided if the failing condition of a patient were detected hours or days earlier.

It should be appreciated that certain types of patient data may become available to the health score system 208 at different times or sampling rates. For example, a heart rate signal from a wearable sensor device 100 may be almost continuously updated, while a caregiver report may only be available once each day, and laboratory data may only be updated every several days. More frequently available patient data may be used to compute a portion of a health score or a partial health score. Such partial health scores may trigger the health score system 208 to collect or request additional patient data to compute one or more full health scores for assessment. Such triggering signals from partial health scores may serve as an adaptive mechanism with respect to the patient data collection specification.

Similarly, caregiver reports may have different timeframes. For example, within a skilled nursing facility, technicians or nursing assistants (such as CNAs) may spend more time with a patient than a nurse (such as an RN). Variations or concerns registered in the patient data supplied by a CNA may trigger a visit from an RN along with an associated caregiver report.

Aspects of the health score system outputs presented herein relate to the development of a general measure of risk for the patient 202, sensitive to a range of patient conditions, available for use by various recipients to assess a patient's state, and more particularly to recognize downtrends which may indicate the onset of a complication.

Various tests, such as blood or urine tests, may be conducted to evaluate a patient and collect patient data. Similarly, the patient 202 may be evaluated or measured by various sensors or instruments that collect raw medical data. Through various patient queries, the patient 202 may be asked about eating habits, mood, vaccinations, drugs taken, problems with walking, the amount of help needed with daily activities, and living arrangements. The patient 202 may be asked a standard series of questions to evaluate mental function. As recorded in caregiver reports, various physical assessments may be performed on the patient 202 to gather information about physiological, psychological, sociological, and spiritual status. A comprehensive patient assessment yields both subjective and objective findings. Subjective findings may be obtained from the health history and body systems review. Objective findings may be collected from the physical examination. Subjective data may be apparent only to the patient affected and can be described or verified only by that patient 202. Pain, itching, and worrying are examples of subjective data. Both subjective and objective information may be captured within the patient data.

Although objective data, such as blood pressure, heart rate, vital signs, or other numerically measurable factors may be used to generate one or more numbers representing overall patient health, subjective data, may be very significant in predicting the general health of the patient 202. Subjective data may be acquired through self-assessments using patient queries and may include warmth of skin, moisture of skin, symptoms of hypotension, chewing, swallowing, manual dexterity, feeling/ appearance of abdomen, bowel sounds, nausea, vomiting, continence, urinary voids, urine color, urine odor, ability to move extremities independently, use of assistive devices, alertness, recognizing persons, orientation to place, orientation to time, speech coherence, pain levels, peripheral vascular warmth, capillary refill, peripheral pulses, edema, numbness, tingling, breath sounds, nail beds, mucous membranes, sputum, cooperation, and so forth. Such factors may be determined through patient queries using a pass/fail, numerical, or letter grade score. Even when the factors may be scored on a pass/fail basis, the transition from passing to failing may be very predictive in indicating the health of the patient 202. For example, if a patient 202 moves from failing two measures, to failing five measures, and then to failing seven measures, the patient 202 may be going through a very serious decline in health, even if vital signs are relatively normal or not changing.

According to certain embodiments, the patient data may incorporate medical data available from an electronic medical record (EMR) system. An EMR may be a computerized legal medical record created within a health organization that delivers care.

The complimentary self-assessment health scoring system may support efficiency in resource allocation. For example, a caregiver may be allocated where most needed using scores and evaluations associated with the technology presented herein. Improving allocation of resources, such as nurses seeing patients, can increase caregiver efficiency. Responding sooner to those patients who are most at risk can increase the effectiveness of the caregivers.

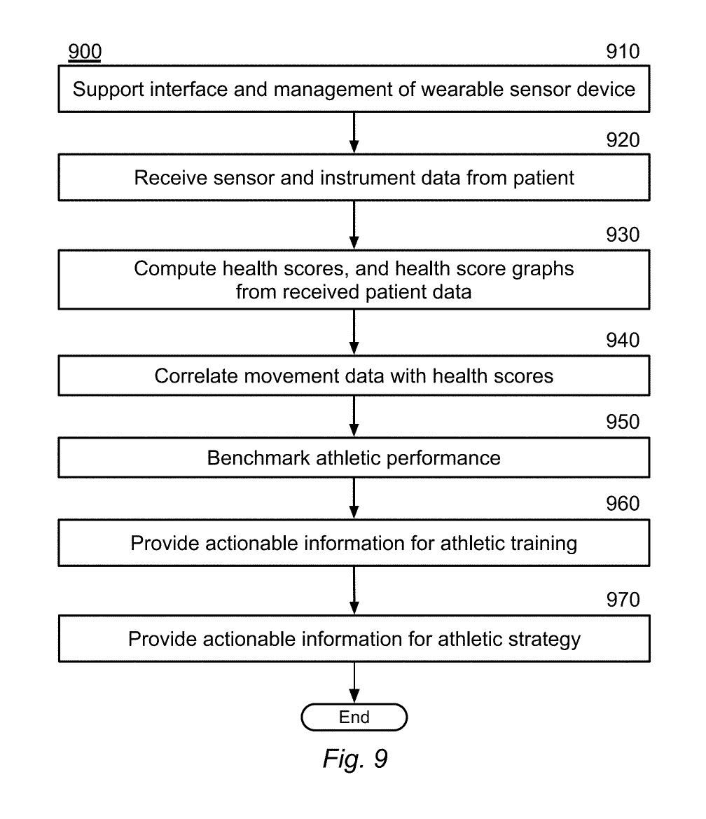

Within the complimentary self-assessment health scoring system, the wearable sensor device 100, the health score system 208, systems associated with patient data, systems associated with the recipients, or any other systems associated with the technology presented herein may be any type of computing machine such as, but not limited to, those discussed in more detail with respect to FIG. 9. Furthermore, any modules (such as the patient device modules 204 or the health score system modules 209) associated with any of these computing machines or any other modules (scripts, web content, software, firmware, or hardware) associated with the technology presented herein may be any of the modules discussed in more detail with respect to FIG. 9. The computing machines discussed herein may communicate with one another as well as other computing machines or communication systems over one or more networks such as network 206. The network 206 may include any type of data or communications network including any of the network technology discussed with respect to FIG. 9.

The health score system 208 may include various health score system modules 209 for generating, interpreting, and presenting one or more health scores and various other health score system outputs. The health score system modules 209 may include an interface module 210, a collection module 220, a sensor calibration module 225, a query module 230, a transformation module 240, a context analytics module 245, a combination module 250, a presentation and comparison module 260, an alert module 270, a recommendation module 280, a storage module 290, and a wearable device management module 295.

The interface module 210 may receive patient data such as signals from the various sensors associated with the wearable sensor device 100. The interface module 210 may be configured to obtain or receive the patient data directly from the wearable sensor device 100 or from other sources as presented herein.

The collection module 220 can aggregate the patient data from the interface module 210. The aggregated data may be provided to the storage module 290. According to certain embodiments, the patient data may be provided to the transformation module 240. The patient data may also be provided to the sensor calibration module 225. The patient data may also be provided to the presentation and comparison module 260.

The sensor calibration module 225 can calibrate and normalize signal data associated with the wearable sensor device 100. The sensor calibration module 225 can process patient data from the interface module 210 and/or the collection module 220 to perform these functions. For example, the accuracy and consistency of systolic and diastolic blood pressure measurements made using sensors such as the PPG and/or Doppler radar may be greatly improved by calibration. Performing such calibration centrally within the health score system 208 can support calibrations over time, calibrations against blood pressure cuff measures from nurses or technicians, and/or calibration analytics over similar patients. Signal data that has been processed by the sensor calibration module 225 may be provided to the transformation module 240 and/or the comparison module 260. The storage module 290 can provide and store historical or analytic data used by the sensor calibration module 225.

The query module 230 may determine the contents and frequency of patient queries. The patient queries may be selected from a database or list of standard queries. Various operational rules may be applied to determine which patient queries should be supplied to the patient 202 and at what times and frequencies.

The transformation module 240 can receive incoming patient data and convert (transform) the data into a usable format for generating one or more health scores associated with the patient. The transformation module 240 can convert each type of patient data into a form that will allow different types of data to be combined. For example, all patent data may be transformed into a scaled value. The transformation module 240 can convert raw patient data into scaled numbers based on various derived transformation functions as presented herein. In one or more embodiments, these transformation functions may be stored within the health score system 208. In one or more embodiments, the transformation functions may be stored in the memory of a separate computer or computing device that is in communication with the health score system 208. In one or more embodiments, the transformation module 240 may convert each type of patient data by operating upon the patient data using one or more excess risk functions that, such as those presented herein. In one or more embodiments, the transformation module 240 may take a past value of one type of patient data, compare it with a current value of the same type of patient data, determine the change in the patient data value, and use the change in value as the input to an excess risk function. In one or more embodiments, the transformation module 240 may take past values of one type of patient data, add it to a current value of the same type of patient data, determine an average value for the patient data, and use the average value as the input to an excess risk function.

As an illustrative example, if a sample of raw patient data collected from the patient 202 is a hemoglobin (Hgb) value corresponding to 10.47 gm/dL, the health score value can be determined by plugging the value of 10.47 gm/dL into a sixth order polynomial function, which represents excess risk due to deviation from normative values, as a function of hemoglobin measured against one-year mortality. This would result in a health score value for hemoglobin of approximately ten percent. Therefore, the transformation module 240 would convert the Hgb value of 10.47 gm/dL to a transformed health score value of ten percent. Similarly, if a sample of patient data collected from the patient 202 is a creatinine value corresponding to 2.5 mg/dL, the health score value can be determined by plugging the value of 2.5 mg/dL into the sixth order polynomial function, which represents excess risk due to deviation from normative values, as a function of creatinine measured against one-year mortality. This would result in a health score value of approximately 31 percent. Therefore, the transformation module 240 would convert the creatinine value of 2.5 mg/dL to a transformed health score value of 31 percent.

The context analytics module 245 can apply contextual analytics to health score metrics including sensor signals associated with the wearable sensor device 100 and metrics derived therefrom. Context may include information such as time since moving, activity levels, calories consumed, sleep state, sleep hygiene, other such contexts, changes in such contexts, and transitions between states associated with contexts. For example, heart rate information may be more meaningful to a health score when put in the context of what the patient is doing while the heart rate is measured. Various sensor measurements made during sleep may indicate entirely different concerns than those made during waking hours.

Application of the health score system 208 in conjunction with the wearable sensor device 100 can support a rich contextual approach to health score analysis. Having the context information of the patient's state or actions at the time of sensors measurement provides an important opportunity to derive meaning from the context. For example, when a patient is walking, a heart rate of 80 BPM may be normal for them, but if they have been sitting still for ten minutes, 80 BPM may mean something completely different. Leveraging a database of the patient's history within the health score system 208, it may be determined whether or not the resting heart rate of 80 BPM is significant. For example, if the patient's resting heart rate has historically been 60 BPM, but a trend over days or months shows an increase to 80 BPM, then an alert or notification should be raised to the patient, their healthcare team, or other recipients.

Data from the transformation module 240 or the context analytics module 245 may then be provided to the combination module 250, which in turn may generate the health score, using a predetermined algorithm. In one or more embodiments, the combination module 250 may take the sum of each of the single-variable risks given by the transformed health scores. The combination module 250 may combine the transformed health score values and scale them, so that they span a given range. According to certain embodiments, when a health score were defined so that a high value corresponded to “good health” and a low value corresponded to “poor health,” then the scaled total transformed health score would be subtracted from the “best” value of health. For example, if the best value were 100, and the range was to be 100, that is poor health was to correspond to zero, then the scaled total transformed health score value would be subtracted from 100. If the scale factor was 0.1 and a health score was computed based just upon these two variables, the two health score variables could be combined (10+31=41) and then the quantity 41 times 0.1 (or 4.1) would be subtracted from 100 and come up with a health score of 95.9. Therefore, the combination module 250 would generate a health score for the patient to be 95.9 at that time. This example describes a health score determination on two types of patient data. Typically, the health score would be determined based on and larger number of types of patent data. This larger number may be 10, 15, 20, 25, 30 or more types of patient data.

The presentation and comparison module 260 may receive the calculated health score and may prepare a health score graph, plotting the health scores for a patient 202 as a function of time. In some embodiments, the presentation and comparison module 260 may render, for display upon a screen or monitor, the health score as a health score graph over a predetermined time frame. The rendered health score graph may be presented to on or more recipients or the patient 202 for visualizing health trends in the patient 202.

The alert module 270 may generate alerts. An alert may be activated when the health score descends below an acceptable threshold or if a downward trend is detected. The storage module 290 may be configured to store and retrieve health score information at various times during the health score generation and presentation processes. The recommendation module 280 may generate recommendations suggesting one or more courses of action associated with patient care. It should be appreciated that an alert may comprise a sampling or history of health scores, any combination of health score outputs, or any notifications or communications associated therewith.

The wearable device management module 295 can provide support to operations of the wearable sensor device 100. For example, user identification, user preferences, associated recipients for the user, charging, maintenance, and so forth.

It should be understood that the illustrated health score system modules 209 are merely one example of the logical organization of modules within the health score system 208. For example, many of the modules may be combined with one another or subdivided and separated according to their function. It should be appreciated that any other modular organization within another health score system 208, employing variously differing logical modules to obtain, interpret, and/or present a health score does not depart from the spirit or scope of the technology presented herein.

Furthermore, it is noted that the modules of the health score system 208 are illustrated to show their logical relationship to one another according to certain embodiments. However, this is not intended to limit the physical construction of such a system. For example, the health score system 208 may be employed on a single larger computer or on a series of smaller computers, possibly with different components residing within different geographical locations, such as the use of an off-site storage module 290. Any other embodiment of such a health score system 208 may employ similar modules to generate a health score alert, as not all embodiments of the present disclosure are intended to be limited in this manner.

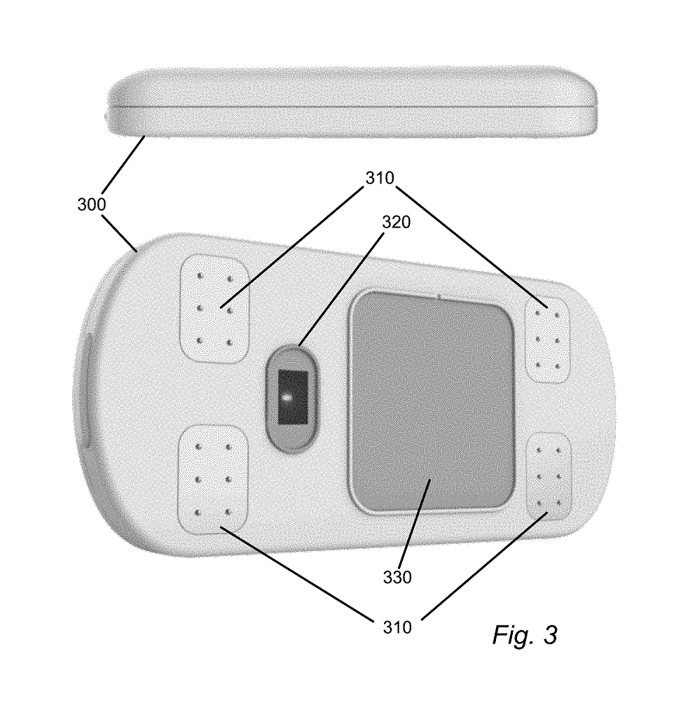

FIG. 3 illustrates two views of an extended remote patch 300, in accordance with certain exemplary embodiments. The extended remote patch 300 can collect and process patient data related to health and wellness. The extended remote patch 300 may comprise one example embodiment of the wearable sensor device 100. Alternatively, the extended remote patch 300 may serve as a peripheral or extension to the wearable sensor device 100.

The extended remote patch 300 may comprise may comprise remote patch electrodes 310. The extended remote patch 300 may comprise four or more remote patch electrodes 310. For example, the extended remote patch 300 may include two electrodes coupled to an internal ECG sensor as well as two electrodes coupled to an internal skin conductance sensor.

The extended remote patch 300 may comprise a photoplethysmogram (PPG) sensor 320. The PPG sensor 320 may use any combination of red, green, infrared, or other color illumination.

The extended remote patch 300 may comprise a remote patch microphone330 for cardiac and respiratory sound sampling.

The extended remote patch 300 may support skin temperature sensing. Skin temperature sensing may share one or more contacts with the internal ECG sensor or the internal skin conductance sensor.

The internal skin conductance sensor may operate using any combination of skin resistance, skin conductance, or galvanic skin resistance (GSR).

The extended remote patch 300 may comprise a gyroscope and/or accelerometer for motion sensing. For example, the extended remote patch 300 may comprise a six axis gyroscope/accelerometer.

The extended remote patch 300 may comprise an internal, rechargeable battery. The extended remote patch 300 may support measurements of ECG, heart rate, respiratory rate, heart rate variability, cardiac pre-ejection period, peripheral capillary oxygen saturation (SPO2), heart sound recording, respiration sound recording, attitude measurement, posture measurement, and/or pedometer functionality for walking or running.

The extended remote patch 300 may comprise Bluetooth, Wi-Fi, or other wireless interface.

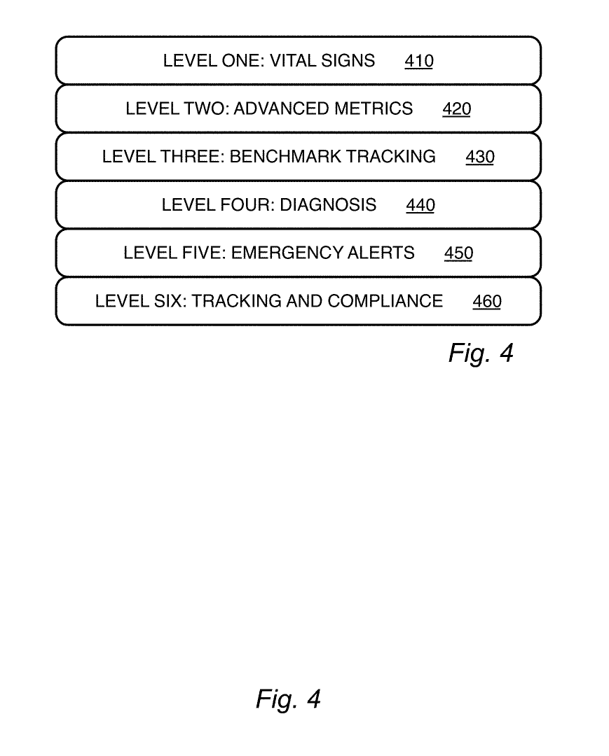

FIG. 4 is a hierarchical diagram illustrating six levels of wearable sensor monitoring functionality, in accordance with certain exemplary embodiments presented herein.

Level one monitoring may include vital signs 410. For example, systolic blood pressure, diastolic blood pressure, heart rate, respiratory rate, blood oxygen saturation, and so forth. SPO2.

Adaptive vital sign monitoring can determine the necessary frequency of vital signs measurement for each individual. Vital signs may be monitored more frequently as their values become a concern.

Level two monitoring may include advanced metrics 420. For example, heart rate variability (HRV), while not a traditional vital sign may be monitored to signal the breakdown of vital bio-feedback mechanisms. As another example, skin conductance, can provide a reliable measure of increasing or decreasing stress. These metrics may be as important, or more important, as traditional vital signs as leading indicators, or predictive indicators of a decline in health. Similar advanced metrics 420 may include mean arterial pressure (MAP), pulse wave velocity (PWV), or other useful metrics that may fall outside of the realm of traditional vital signs but may still be accessibly via the wearable sensor device 100 and/or the extended remote patch 300.

Level three monitoring may include benchmark-tracking 430. For example, the wearable sensor device 100 can utilize movement sensors such as 3-axis accelerometer, gyroscope, altimeter, 6-axis sensor, and so forth to tag each medical measurement. For example, when sensing heart rate, it may be determined if it is a resting heart rate or not. New metrics may be established when gain meaning through the tracking of a group or an individual, according to data analytic techniques. Without bringing a patient into a doctor's office every day for a treadmill test, changes to the HR, SBP, SPO2, RR can be continuously, or frequently, monitored as the patient goes about his or her usual business. Defining and tracking patient metrics through data analytics techniques can automatically establish self-normalized, early detection of health score decline.

Level four monitoring may include diagnosis 440. For example, the wearable sensor device 100 may diagnose atrial fibrillation, obstructive sleep apnea, congestive heart failure, diabetes, or other disorders. In addition to atrial fibrillation, arrhythmia, tachycardia, and the like, diagnosis possibilities expand with the availability of data sets concerning all major chronic diseases as the use of the wearable sensor device 100 expands across an increasingly varied population.

Level five monitoring may include emergency alerts 450. For example, the wearable sensor device 100 may predict or detect heart attack, stroke, fall, or other emergencies. The wearable sensor device 100 may also call for emergency help over a mobile or other communications network. The emergency call may be subject to voice or button cancellation by the user. A warning may be announced and a n internal microphone activated from which the user can cancel the emergency call or redirect the call elsewhere. According to certain examples, in the absence of a verbal or physical response, help may be automatically summoned.

Level six monitoring may include tracking and compliance 460. For example, once a diagnosis has been given, compliance or tracking may be adapted. For example Parkinson tremors may be detected. Given a diagnosis, there may be specific symptoms that could be tracked as indicators of response to treatment, compliance with prescriptions, or progression of disease.

Preventive or coping intervention may be supported. For example, detect an imminent migraine, and then activate counter-measures such as anti-seizure stimulation. Another example, COPD might be tracked by the lung sounds or by decrease in heart function. Another example, for Parkinson's disease, steadiness of gait and amount of arm tremor may be measured.

### Example Graphs and Data Structures

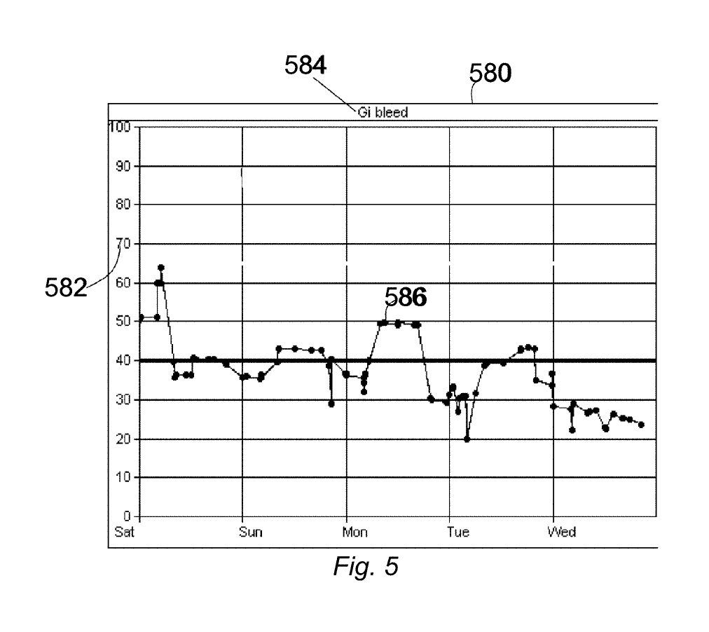

FIG. 5 is a chart 580 showing a health score graph, in accordance with certain exemplary embodiments presented herein. The illustrated health score graph plots a series of health scores, calculated by the health score system 208, as a function of time. The chart 580 includes scale markings 582, a heading label 584, and a plot 586. The plot 586 is one example health score graph for a patient recovering from a gastrointestinal (GI) bleed. During a GI bleed, the amount of bleeding can range from nearly undetectable to acute, massive, and life threatening. At the beginning of plot 586 (which is the first of the five days represented), the patient had a health score in the low 50s. Shortly thereafter, the patient's health score improved to a value in the 60s. However, at some point on the first day, the health score began to decline quickly from a value in the 60s to a value in the 30s. Accordingly, at the middle of the first day, the health score graph can prove to be a critical tool for medical care. The illustrated health score graph may make it very clear to a recipient that something is going wrong with the patient 202 at the end of day one. This may be a critical time for the patient 202 where immediate treatment may prevent a crisis.

As described herein, health scores for a patient 202 may be generated by computing excess risk due to deviation from normative values, as a function of each type of medical data measured against one-year mortality. To provide the health score with continuous input functions for each type of medical data, average one-year mortality versus averages of each type of medical data for distinct ranges, may be fit to higher-order polynomials based on data obtained from an EMR for a plurality of different patients.

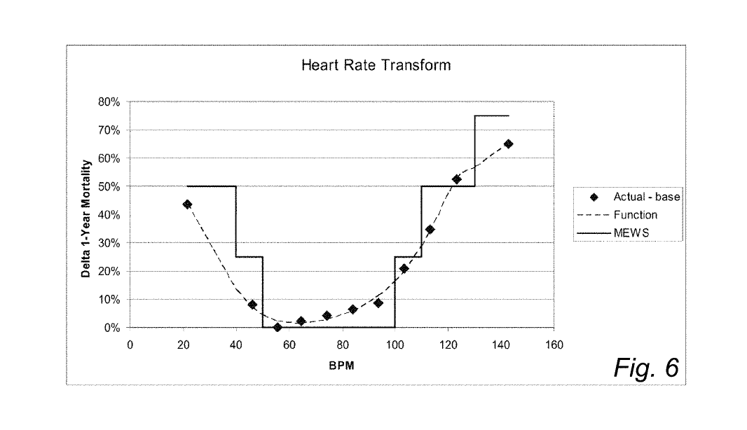

FIG. 6 is a graph of an excess risk curve where an outcome measurement is a function of heart rate, in accordance with certain exemplary embodiments. The outcome measurement is a likelihood of mortality one year later. The medical data was obtained from EMR data from about 22,000 patients. A polynomial (such as a sixth order polynomial) may be constructed to define the excess risk due to deviation from normative values. The excess risk curve shows that a heart rate value of approximately 55 BPM (conventionally considered in the range of “well-trained athletes”) results in a zero percent excess risk for a patient, however a heart rate value of approximately 92 BPM (conventionally considered in the high range of a “normal value”) results in an about nine percent excess risk for a patient. Also illustrated is a Modified Early Warning System (MEWS) curve for heart rate, which identifies people at risk of deterioration in a busy hospital ward. The MEWS curve contains a risk transform in the form of a step function: output=2 if HR<40, output=1 if HR is between 40 and 50, output=0 if HR is between 51 and 100, output=1 if HR is between 100 and 202, output=2 if HR is between 202 and 130, and output=3 if HR is greater then 130. The step function is plotted by setting 1 point=25%. As illustrated graphically, there is a correspondence between the two transformation curves (MEWS) and the excess risk curve of the function even though these may be derived completely independently. The MEWS step-function curve comes from the accumulated observations of experts in the field. In this case doctors having witnessed many patients go through crises.

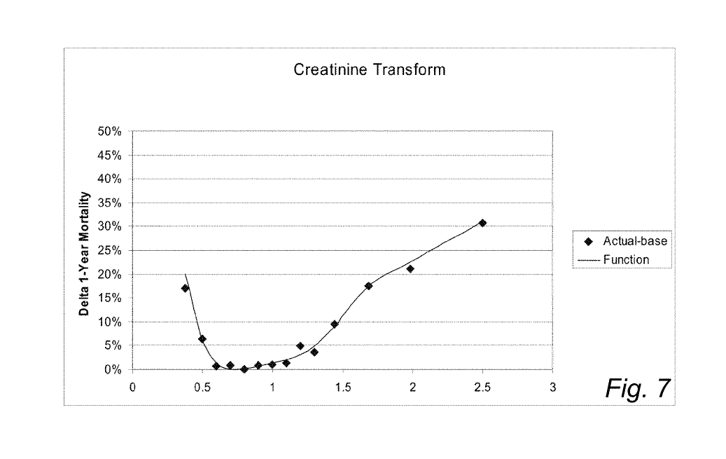

FIG. 7 is a graph of an excess risk curve where an outcome measurement is a function of creatinine, in accordance with certain exemplary embodiments. The outcome measurement is a likelihood of mortality one year later. The medical data was obtained from EMR data from about 22,000 patients. A sixth order polynomial (fit in the range of 0.37 mg/dL to 2.5 mg/dL) defining the excess risk due to deviation from normative values for bicarbonate was derived. The excess risk curve shows that a creatinine value of approximately 0.8 mg/dL conventionally considered a “normal value”) results in a 0% excess risk for a patient, a creatinine value of approximately 2.5 mg/dL (conventionally considered a “high value”) results in an about 31% excess risk for a patient, and a creatinine value of approximately 0.37 mg/dL (conventionally considered a “low value”) results in an about 17% excess risk for a patient. If a creatinine value for a different patient is less then 0.37 mg/dL, then delta-one year mortality will be equal to 20%. If a creatinine value for a different patient is more then 2.5 mg/dL, then delta-one year mortality will be equal to 30%.

In yet another example of an excess risk curve (not illustrated here), an outcome measurement (mortality one year later) is a function of a continuous variable (bicarbonate). The medical data was obtained from EMR data from about 22,000 patients. A 6th order polynomial defining the excess risk due to deviation from normative values for bicarbonate was derived. The excess risk curve shows that a bicarbonate value of approximately 24 mEq/L (conventionally considered a “normal value”) results in a 0% excess risk for a patient, however a bicarbonate value of approximately 13 mEq/L (conventionally considered a “low value”) results in an about 33% excess risk for a patient.

In yet another example of an excess risk curve (not illustrated here), an outcome measurement (mortality one year later) is a function of a continuous variable (hemoglobin). The medical data was obtained from EMR data from about 22,000 patients. A 6th order polynomial defining the excess risk due to deviation from normative values for bicarbonate was derived. The excess risk curve shows that a hemoglobin value of approximately 15.5 gm/dL (conventionally considered a “normal value”) results in a 0% excess risk for a patient, however a hemoglobin value of approximately 7.64 gm/dL (conventionally considered a “low value”) results in an about 13.1% excess risk for a patient.

In yet another example of an excess risk curve (not illustrated here), an outcome measurement (mortality one year later) is a function of an ordinal score (Braden Scale). Medical data used to derive the function was obtained from EMR data from about 22,000 patients. Points on the graph are average one-year mortality for a given Braden scale score less base mortality for a Braden score of 23 (base mortality is 2.6% for a Braden score of 23). Average one-year mortality versus average Braden scale score for distinct ranges were fit to a 5th order polynomial.

In certain embodiments, the type of medical data that is collected is a categorical class. An example of a categorical class includes, but is not limited to, a heart rhythm distinction. In an embodiment, the heart rhythm distinction includes sinus bradycardia, sinus rhythm, heart block, paced, atrial fibrillation, atrial flutter, sinus tachycardia and junctional rhythm. In an embodiment, the type of medical data that is collected is a binary assessment. An example of a binary assessment is subjective data, such as nursing assessments. In an embodiment, the binary assessment is a nursing assessment. In an embodiment, the variable selected from the nursing assessment includes, but is not limited to, a food assessment, a neurological assessment, a psychiatric assessment, a safety assessment, a skin assessment, a genitourinary assessment, a muscular-skeletal assessment, a respiratory assessment, a cardiac assessment, a peripheral vascular assessment, a gastrointestinal assessment, a Braden scale assessment and a pain assessment.

In yet another example of an excess risk curve (not illustrated here), an outcome measurement (mortality one year later) is a function of a categorical class (heart rhythm distinction). The medical data was obtained from EMR data from about 22,000 patients. The excess risk curve shows that a heart rhythm of sinus bradycardia (conventionally defined as a heart rate of under 60 BPM) results in a 0% excess risk for a patient. A heart rhythm of atrial fibrillation (conventionally defined by the quivering of the heart muscles of the atria) results in a 21% excess risk for a patient. A heart rhythm of junctional rhythm results in a 29% excess risk for a patient.

In yet another example of an excess risk curve (not illustrated here), an outcome measurement (mortality one year later) is a function of a binary assessment (food/nutrition nursing assessment). Medical data used to derive the function was obtained from EMR data from about 22,000 patients. The excess risk curve shows that if a patient has failed a food/nutrition nursing assessment, there is a 43% excess risk for a patient.

It should be understood that although the embodiments described with respect to these examples of excess risk curves, functions are derived based on a single independent variable (type of medical data), the methods disclosed herein are also applicable for deriving functions based on two or more independent variables (types of medical data). As with functions of one variable, functions of several variables can be represented numerically (using a table of values), algebraically (using a formula), and sometimes graphically (using a graph).

According to various embodiments, a general measure of risk for a patient, sensitive to the full range of patient conditions, independent of diagnosis, is developed which can be used to assess a patient's state, and more particularly to system and methods for recognizing downtrends which may indicate the onset of a complication. Certain embodiments of the present inventions provide systems and methods for recognizing downtrends in a patient's health, which may indicate the onset of a complication, and to aid in communication of this information across staff handoffs. At least some of the embodiments allow for continually tracking the health of a patient in their location (home, nursing home, hospice, hospital, rehabilitation center, and so forth). At least some of the embodiments allow various caregivers to provide more effective health care for each patient. In addition or alternatively, at least some embodiments assist caregivers in avoiding errors and reducing crisis management by using the systems' capability to detect trends in a patient's health before the patient reaches a crisis point. Recognizing a decline soon enough to administer proper treatment may be a life-saving benefit. Embodiments of the system may give caregivers a way in which to get the “big picture” of a patient's condition and absorb in a glance perhaps 100 pages of a patient's medical records. This deeper understanding, along with this new capability to detect health trends, short-term (over the space of hours) and/or long-term (over the space of days) may be important in delivery of effective medical care. Embodiments may enable a new field of scientific study, where medical and surgical treatments can be evaluated by the new measurements provided by embodiments of the present disclosure.

Various embodiments of the present disclosure may generate a patient health score. The health score may be continually plotted and displayed to show each patient's medical progress during his hospital stay. The health of the patient may relate a patient's vitality and overall quality of life rather than simply being free from disease. Although a patient who has a terminal disease, such as cancer, may conventionally be considered to be in poor health; however, if a cancer patient who only has a few months to live is playing a game of ping-pong for hours, he/she may be considered to be in good health, as the term is used herein. In comparison, a patient who entered the hospital to have a simple surgery, such as a tonsillectomy, may conventionally have been considered to be and will likely recover to be in excellent health. However, while recovering, the tonsillectomy patient's vitality might be low and his/her chance of dying in the near future could be much higher if a complication were to arise; thus, the patient may be considered to be in poor health, as the term is used herein. The health of a patient may relate to the patient's overall physical, mental, spiritual and social wellbeing and not merely the absence of disease or infirmity. Embodiments of the present invention may significantly improve the quality and continuity of medical care.

In addition to the features of the health score and uses thereof, it is further contemplated that an exemplary use of such system may include the use of the health score to provide a panel of health score charts, providing caregivers an overview as to the progress of many patients at one time, as is described further below.

In one embodiment, the health score may be used to predict the odds of a crisis within N number of hours. That is, for example, there is a 20% chance of a crisis in the next 12 hours. This information may be used to assign additional observation to particular patients, or if a crisis is judged to be imminent, a call may be initiated to a Rapid Response Team. Another use for the health score is to schedule caregiver visits. This will allow the caregiver to quickly move to patients requiring more attention first, and then proceed to less critical patients.

One way in which a crisis may be predicted is by comparing the individual patient's health score with a standard recovery or wellness curve. By tailoring the standard recovery curve to the patient, better results may be obtained. For example, one of the exemplary ways in which patients may be categorized is by DRG/ICD-9 grouping systems. DRG stands for a diagnostic related group and ICD-9 is the international classification of disease. Both of these are ways of categorizing patients based on what disease or ailment the patients have and are employed by insurance companies to figure out how much the insurance company should pay out for a particular policyholder. For example, the standard recovery curve for someone having had elective rhinoplasty is likely to be very different from the standard recovery curve of someone who had a heart-lung transplant. If the rhinoplasty patient's health was declining, but the rhinoplasty patient's health was viewed in comparison with someone who had serious surgery, such as a heart-lung transplant, the decline might not be viewed as being significant, while in reality the rhinoplasty patient could be about to experience a cardiac or respiratory crisis. If the transplant patient's health is improving, but the patient's health is viewed in comparison with other patients who have had the same procedure and the recovery is much slower this could be an early indication of a complication. By comparing patients based on their disease, treatment/surgery, or affliction, the patient's health score may be better interpreted.

In some embodiments, ICD-9, which groups patients into thousands of detailed categories, normative data plots may be used, while in some embodiments DRG, which groups patients into about 500 categories, may be used, while in yet other embodiments, a combination of the two grouping systems may be used. Not all embodiments are intended to be limited in this respect and any disease grouping system or data may be employed to create a singular or combination standard recovery curve.

According to various embodiments, creating the standard curve may entail reviewing graphs of all previous patients with the same DRG/IDC-9 code in a database and plotting them as one or more curves. The curve may be represented by an average curve, all of the individual patient's curves, a median curve, a top 25th percentile and a bottom 25th percentile, plus or minus some number of standard deviations thereby creating a normative recovery as well as upper and lower bounds, any combination of the foregoing or any other representative indicator as not all embodiments of the present disclosure are intended to be limited in this respect. By using these types of normative curves a doctor may be able to see that even if a patient is recovering, the patient might be recovering more slowly (too shallow a slope) than the average patient with a similar condition and this slower recovery might be cause for further investigation.

Not only may the grouping codes be useful in comparison with the health score, but the grouping codes may be utilized in generating a more accurate health score. In some embodiments, a user may modify the algorithm used to generate the health score based on the diagnosis or grouping code of the patient in order to have the health score more accurately reflect the patient's recovery

In some embodiments, the life expectancy or mortality of a patient, such as the likelihood that a patient will die within the next 24 hours, may be predicted. For example, if a terminal patient is listed as DNR (do not resuscitate) or “keep patient comfortable,” a family member may want to know the life expectancy of the terminal patient to plan for the inevitable death.

By comparing a patient's health score with a standard, many inferences may be drawn from the comparison. For example, in some embodiments, patients may be given a category, such as critical, critical but stable, serious, serious but stable, fair, and/or good. These categories may be words or terms, numbers (such as 1-5 or 1-100), colors (such as red, orange, yellow, or green), a made up system of categorizing, or any other system. In addition, the categories may be discrete, such as choosing one of four colors or may be continuous, such as choosing any number from one to 100.

By having patients categorized, administrative decisions and care priority can be determined accordingly. For example, in some embodiments, a nurse scheduling tool may be incorporated or separately determined which would allow shift nurses to see the conditions of all patients on the floor and assign nurses based on skill level, so that more experienced nurses have more critical patients and newer nurses have more stable patients. In some embodiments, the nurse scheduling tool may rank patients, for example, 1-10 and allocate patients to each nurse so that no nurse has a total patient rank of for example, more than 25 (e.g., two very critical patients of rank 10 and one fair but stable patient of rank 5, four fair but stable patients of ranks 5.2, 5.4, 5.7 and 6.1, or two serious patients of rank 8 and one serious but stable patient of rank 7.2). In some embodiments, the ELOS prediction may be incorporated into the nursing schedules, so that discharges may be predicted and the charge nurse may be able to know how many staff members may be required to work an upcoming shift. Similarly, these systems may be applied to routing a doctor's rounds, as described above.

In another possible arrangement, the health score may be used to determine priority and timing of the post-discharge “how are you doing” call. For example, patients leaving the hospital with favorable heath scores may be called in three days for a checkup, whereas patients with marginally acceptable heath scores may be called sooner.

The heath score as disclosed in the incorporated documents, and above may be fine-tuned to each hospital in which it is implemented. Most hospitals have slight differences in procedures, standards, requirements and other elements of daily practice as compared to other hospitals and some embodiments of the present disclosure may be adapted to a specific hospital's preferences. In particular, when using subjective variables to produce a health score, as will be described further below, some hospitals may be more conservative in evaluating a patient's condition. For example, nurses at a first hospital may be taught that slightly grey skin is a reason to fail a skin assessment while nurses at a second hospital may be taught that a patient should pass a skin assessment until the skin is really grey. This difference may make average scores on the health score lower at the first hospital, which could mean that the predicted health of a patient would appear worse at the first hospital than at the second hospital. By adjusting the health score according to an individual hospital's procedures, the health score may be more accurate.

In some embodiments, the heath score may be used for evaluation purposes. For example, the health score may be used to evaluate the performance of a particular caregiver, or even of a care facility. It can also be used to evaluate a particular treatment by studying heath score charts of patients that underwent a particular treatment.

In addition to evaluation of caregivers, the system may be used to compare effectiveness of medical treatments, compare the quality of care provided by different wards or facilities, and compare the skill of healthcare providers by providing an objective assessment of a patient's health and response to various factors. In some embodiments, the algorithm may be customized after a patient's stay to further evaluate the care of the patient and compare the patient with other patients. For example, if two patients had the same diagnosis and received different treatments, a facility or caregiver may want to compare those two patients' recoveries. However, if one patient had a small drop in their health score due to an unrelated event, such as having an allergic reaction to topically applied medication, the algorithm may be adjusted to exclude a factor, such as a skin standard of the nursing assessment, from the health score of both patients, so that the two patients are still evaluated using the same algorithm, but the comparison is tailored to focus on the recovery from the treatments and exclude unrelated deviations.

In another embodiment, the health score chart shapes can be clustered to discover the “types” of patient health trajectories. General prototypical trajectories, or trajectories computed as a function of disease or procedure may be compared against actual health score charts to determine how a particular patient is responding to treatment. Once a health score chart is assigned to such a prototypical trajectory, it may further indicate the likelihood of various outcomes. In some embodiments, this may be accomplished by using DRG/IRC-9 groupings, as discussed herein.

In another embodiment of the present disclosure, the health score may be used as part of a remote monitoring service, where a remote health service provider can monitor the score of several patients and alert an on-site staff if there is an emergency. The health score can be refined using neural networks, or other analytical methods. The health score may be fed to a central data hub and be used to monitor for large-scale trends in health problems, including a biological or chemical attack.

While in some embodiments an individual health score falling below a minimum mark or the change in health score or slope of the health scores falling below a minimum change may trigger an alarm or be interpreted by a healthcare provider as an indication of the patient's declining health, in some embodiments the change in slope or derivative of the slope of the health scores falling below a certain minimum may trigger an alarm or be interpreted by a healthcare provider as an indication of the patient's rapidly declining health. For example, if a patient is slightly declining and suddenly starts to decline at a much faster rate, this change in the acceleration of the slope may trigger an alarm. In some embodiments, the curvature of the health score plot may be provided, such as by a presentation and/or comparison module.

Many times a patient's health may be compromised in favor of conforming the patient's care to hospital standards. For example, taking a patient's vital signs every two-to-four hours generally requires awakening patients during the night and often times not allowing them to complete a full sleep and enter deep sleep, which may be critical to a patient's recovery, and to draw blood from patients every day or two, which can be detrimental to an anemic or hemophiliac. If a patient has been recovering well and has an increasing health score, a healthcare worker may rely on the health score to determine whether or not a routine test or procedure may be skipped in order to allow the patient to better recover.

The system may include the ability to view a patient's prior visits to hospitals or other relevant facilities. In some embodiments, if a patient has a recurring condition, it may be preferable to view that patient's past health scores in addition to the present health score. In addition or alternatively, the graph may display a one or more health scores calculated using different inputs, such as a red line with circular data points for when the entry reflects nursing assessments, a blue line with square data points for blood work and/or a green line with triangular points for a chem panel. Differences in data source may be represented with unique icons or any other means of differentiating them, as not all embodiments are intended to be limited in these respects. In addition or alternatively, a caregiver may click on or hover over a point to access additional information, such as the data inputted to calculate the health score, an average reading, values from earlier in the patient's stay, or any other information.

In some embodiments of the present disclosure, a health score system is provided for generating and presenting a health score chart. The health score may be a medical reference “figure-of-merit” that is used by a health caretaker, such as a physician, nurse, or other health attendant, to track the patient's health before, during or after a medical procedure or illness, in order to assist in preventing that patient from reaching a health crisis. When used in this manner, the health score chart enables the attending physicians and nurses to detect trends in the patient's health over time. The health score chart also provides a statistically significant “outcome” for both clinical studies and retrospective studies of the relative efficacies among various surgical procedures or techniques, and among medical treatments and drugs.

In addition to short-term intensive use of the health score system, a similar modified form may be used on a long-term basis by regular general practitioners or other health care facilitates such as nursing homes. For example, as it stands, yearly physicals are usually accompanied by a series of medical measurements of the patient. Entering such data in health score system may be useful in spotting long term declining health trends, even if none of the particular medical conditions have reached a crisis level.

### Example Processes

According to methods and blocks described in the embodiments presented herein, and, in alternative embodiments, certain blocks can be performed in a different order, in parallel with one another, omitted entirely, and/or combined between different example methods, and/or certain additional blocks can be performed, without departing from the scope and spirit of the invention. Accordingly, such alternative embodiments are included in the invention described herein.

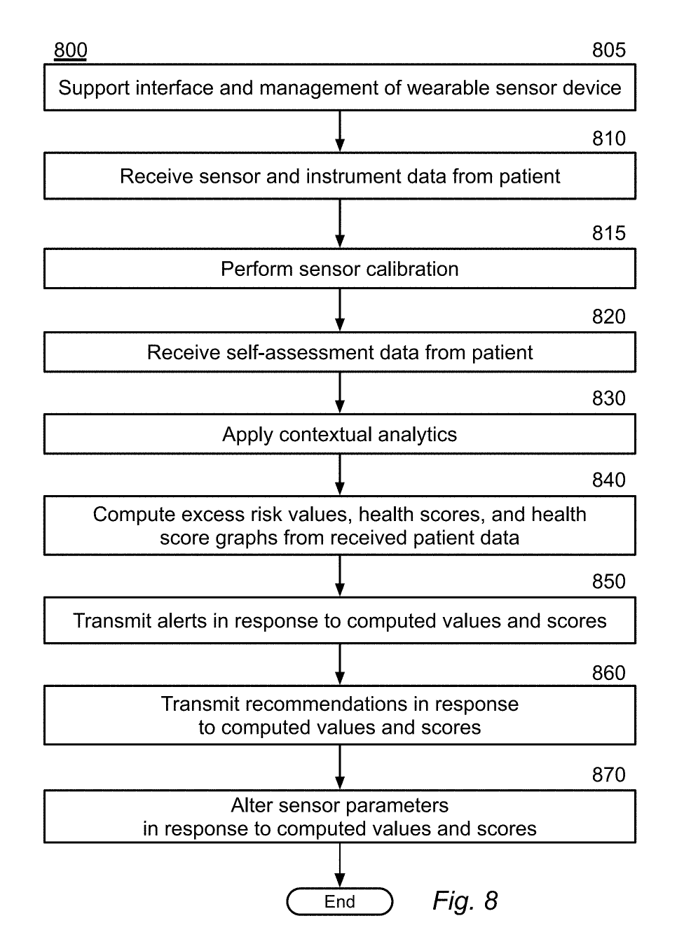

FIG. 8 is a block flow diagram depicting a method 800 for applying wearable sensor data to adaptive health score tracking, in accordance with certain exemplary embodiments. In block 805, interfacing and management of the wearable sensor device 100 may be supported. Sensor data collection as well as user interfacing may be supported between the health score system 208 and the wearable sensor device 100.

In block 810, data may be received from the wearable sensor device 100, other sensors, and/or instruments associated with the patient 202. The interface module 210 may receive patient data such as signals from the various sensors associated with the wearable sensor device 100. The interface module 210 may be configured to obtain or receive the patient data directly from the wearable sensor device 100 or from other sources as presented herein. The collection module 220 can aggregate the patient data from the interface module 210.

In block 815, the sensor calibration module 225 can calibrate and normalize signal data associated with the wearable sensor device 100. The sensor calibration module 225 can process patient data from the interface module 210 and/or the collection module 220 to perform these functions.

In block 820, self-assessment data may be received from the patient 202. The self-assessment data may include patient query responses to health and wellness related patient queries. The patient queries may seek to ascertain how the patient feels subjectively, how energetic or vital the patient is, the patients quality of life, how the patient is eating and sleeping, how the patient is complying with medications and/or therapies, how the patient rates their perception of pain or discomfort, and so forth. These patient queries may be administered by voice telephone call, instant messaging, text messaging (SMS), television set top box, networked appliance, voice conference, video conference, in person, via web site, software application, mobile (smartphone/tablet) application, wearable computing device (smart watch), smart appliance, or through any other communication device or computing machine.

In block 830, the context analytics module 245 can apply contextual analytics to health score metrics including sensor signals associated with the wearable sensor device 100 and metrics derived therefrom. Context may include information such as time since moving, activity levels, calories consumed, sleep state, sleep hygiene, other such contexts, changes in such contexts, and transitions between states associated with contexts.

Various other contextual data may include indoor motion sensors, door/window switches, patient-motion sensors, accelerometer, pedometer, and so forth. Such sensors can detect how much a patient is moving around their neighborhood, house, or room, how frequently they go to the kitchen (as a proxy for how well they are eating), how frequently they visit the restroom, how often they leave the house, how many steps they take each day, how restfully and how long they sleep, and so forth. Instruments for additional sensors or contextual information may include heart monitors, blood oxygen monitors, respiration monitors, blood/urine test machines, oxygen tanks or concentrators, insulin pumps, temperature sensors, weight scales, blood pressure cuffs, pain medication pumps, or any other medical or physiological monitors or instruments.

The contextual patient data may include caregiver reports incorporating information reported from various caregivers o other recipients. The contextual patient data may also include lab results and medical data such as that from one or more EMR systems.

In block 840, excess risk values, health scores, and health score graphs may be computed from received patient data. The various pieces of patient data received in blocks 810, 815, 820, and 830 may be scaled and/or combined and optionally combined with supplementary patient data (such as chart data, medical records data, and previous computed values) to compute various excess risk values, composite excess risk values, combined health scores, and graphs/plots of health score trends over time.

In block 850, alerts may be transmitted to one or more recipients in response to the computed values or scores from block 840 crossing a specified (or computed) threshold or in response to the computed values taking on a negative trend, which may indicate an overall decrease in the patient's health outlook.

In block 860, recommendations may be transmitted to the patient 202 and/or one or more recipients in response to the computed values or scores from block 840 crossing a specified (or computed) threshold or in response to the computed values taking on a negative trend, which may indicate an overall decrease in the patient's health outlook. The recommendations may include: sending a nurse or technician to the patient location, recommending a visit to a doctor or laboratory, recommending behavior changes (e.g., more sleep, dietary changes, more exercise), and/or recommending relocating the patient to another facility/location (e.g., discharge to home, nursing home, hospice, hospital admission, readmission, rehabilitation facility, an so forth).

In block 870, sensor parameters may be modified in response to the computed values. Parameters may include sample rates, calibration settings, resolution, and so forth. Self-assessment parameters (such as patient data collection specifications) may also be modified in response to the computed values.

Sensor parameters may be modified in response to scores from block 840 crossing a specified (or computed) threshold or in response to the computed values taking on a negative trend which may indicate an overall failing in the patient's health outlook. Modifying the sensor parameters may increase, decrease, or terminate sensor queries or the collection of patient data in order to obtain addition patient information. Modifying the parameters may update the type or frequency of sensor measurements for the collecting of patient data. Modifying the parameters may also terminate one or more patient monitoring or data collection functions as a patient recovers or other changes occur.

FIG. 9 is a block flow diagram depicting a method 900 for applying wearable sensor packages and health score analytics to athletic performance and training, in accordance with certain exemplary embodiments. In block 910, interfacing and management of the wearable sensor device 100 may be supported. Sensor data collection as well as user interfacing may be supported between the health score system 208 and the wearable sensor device 100. It should be appreciated that the athlete may be a human subject or another type of animal, such as a race horse.

In block 920, data may be received from the wearable sensor device 100, other sensors, and/or instruments associated with the patient 202. The interface module 210 may receive patient data such as signals from the various sensors associated with the wearable sensor device 100. The interface module 210 may be configured to obtain or receive the patient data directly from the wearable sensor device 100 or from other sources as presented herein. The collection module 220 can aggregate the patient data from the interface module 210.

In block 930, health scores, and health score graphs may be computed from received patient data. The various pieces of patient data received in block 920 may be scaled and/or combined and optionally combined with supplementary patient data (such as chart data, medical records data, and previous computed values) to compute various excess risk values, composite excess risk values, combined health scores, and graphs/plots of health score trends over time.

In block 940, movement data received in block 920 may be correlated with health scores and related sensor information. Data analytic techniques may be applied to physiological measurements along with motion, velocity, and acceleration data.

In block 950, athletic performance may be benchmarked. Changes, such an deterioration or improvement, in performance man be correlated to changes in physiological sensory data from the wearable sensor device 100.

In block 960, actionable information for athletic training may be provided according to collected sensor data, movement data, or benchmarking results. Actionable information for athletic training may be also provided according to any analysis or data analytics performed on collected sensor data, movement data, or benchmarking results. For example, if training is most effective at night, or before eating, or when broken up by frequent recovery periods, these analysis results may be useful for the training of the specific athlete in question.

In block 970, actionable information for athletic strategy may be provided according to collected sensor data, movement data, or benchmarking results. Actionable information for athletic strategy may be also provided according to any analysis or data analytics performed on collected sensor data, movement data, or benchmarking results. For example, if speed performance is improved after training in certain metric regimes, while endurance is improved in others, these analysis results may be useful for establishing strategies for the specific athlete in question.

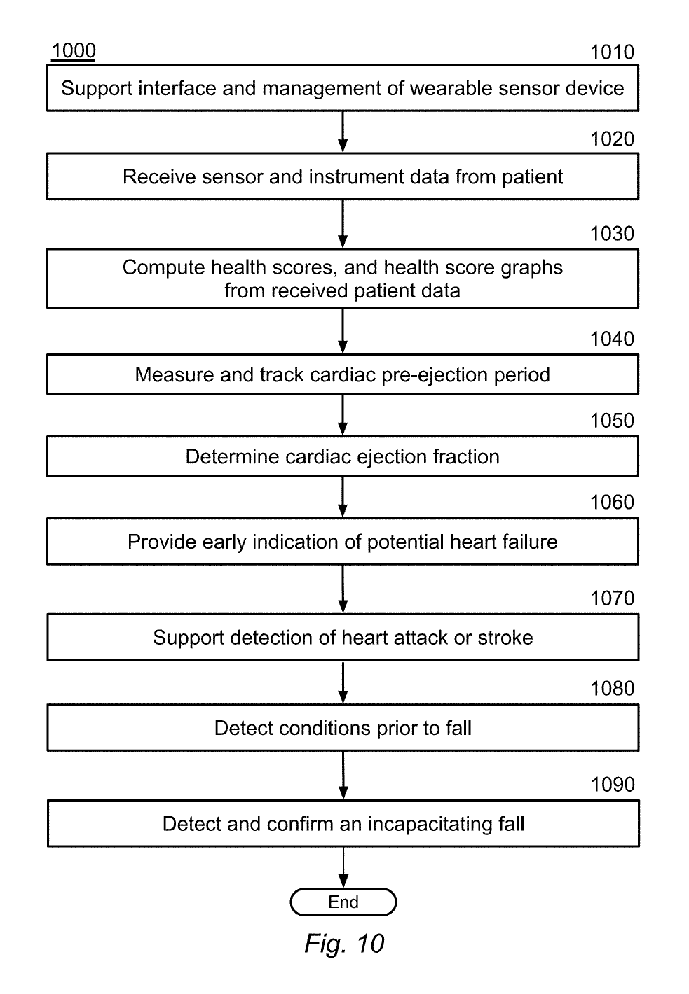

FIG. 10 is a block flow diagram depicting a method 1000 for applying wearable sensor packages and health score analytics to the prediction and detection of adverse health events, in accordance with certain exemplary embodiments. In block 1010, interfacing and management of the wearable sensor device 100 may be supported. Sensor data collection as well as user interfacing may be supported between the health score system 208 and the wearable sensor device 100.

In block 1020, data may be received from the wearable sensor device 100, other sensors, and/or instruments associated with the patient 202. The interface module 210 may receive patient data such as signals from the various sensors associated with the wearable sensor device 100. The interface module 210 may be configured to obtain or receive the patient data directly from the wearable sensor device 100 or from other sources as presented herein. The collection module 220 can aggregate the patient data from the interface module 210.

In block 1030, health scores, and health score graphs may be computed from received patient data. The various pieces of patient data received in block 1020 may be scaled and/or combined and optionally combined with supplementary patient data (such as chart data, medical records data, and previous computed values) to compute various excess risk values, composite excess risk values, combined health scores, and graphs/plots of health score trends over time.

In block 1040, cardiac pre-ejection period (PEP) may be measured and tracked. Cardiac pre-ejection period (PEP) is the time interval after start-of-compression within the left ventricle until the aortic valve opens to allow blood flow out of the left ventricle into the Aorta. The start-of-compression within the left ventricle may be considered the arrival of an electric signal. PEP is inversely related to cardiac ejection fraction (EF). PEP may be measures from wearable sensor device 100 and incorporated into associated health scores.

In block 1050, cardiac ejection fraction (EF) may be determined. EF may be cited as a percentage representing the fraction of left ventricle blood capacity that flows into the Aorta. EF is the primary measure used by cardiologists to judge heart function. Normal EF is about 65% and a patient may be considered to be in heart failure when EF falls below 40%. Traditionally, PEP and EF are not regularly measured or recorded since doing so requires an echocardiogram to be administered.

In block 1060, early indication of potential heart failure may be supported. Tracking PEP can support determining heart failure earlier than traditional symptoms would support. Accordingly, a patient could be examined and treated before symptoms would otherwise cause the patient to seek medical care. Congestive Heart Failure (CHF) may be progressive, irreversible, and fatal. CHF can progress for years before symptoms are noted. Detection of CHF at an early stage, before permanent damage to the heart, provides an opportunity to avoid increasingly negative outcomes. Lifestyle changes may be sufficient or open other opportunities for effective treatment.

In block 1070, detection of heart attack or stroke may be supported by sensor measurements received from the wearable sensor device 100 or from associated data analytics.

In block 1080, detection of conditions prior to a patient fall event may be supported by sensor measurements received from the wearable sensor device 100 or from associated data analytics.

In block 1090, detection and confirmation of an incapacitating fall event may be supported by sensor measurements received from the wearable sensor device 100 or from associated data analytics.

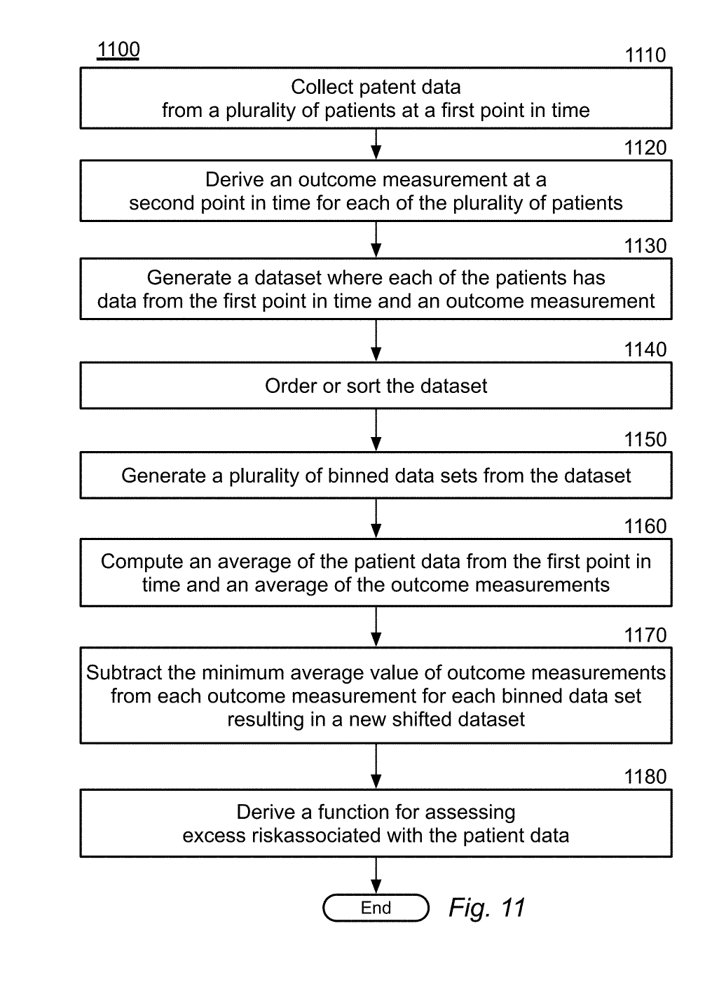

FIG. 11 is a block flow diagram depicting a method 1100 for assessing risk associated with at least one type of patient data due to deviation from normative values, in accordance with certain exemplary embodiments.

In block 1110, patient data may be collected from a plurality of patients 202 at a first point in time. The collected patient data may be the same type of medical data for each of the plurality of patients 202. According to various embodiments, the first point in time can correspond to when the patient enters a stage of care (e.g., at discharge from a hospital, or entering a nursing home). According to various embodiments, the type of patient data that is collected may be represented as a continuous variable, an ordinal score, a categorical class, a discretized and/or binary assessment value. According to certain embodiments, the patient data may be collected from an electronic medical record (EMR). According to certain embodiments, the patient data may be all or in part from self-assessed patient query responses. The self-assessed patient query responses may be made in response to patient queries that are adaptively generated by an automated health score system 208 complimented by one or more human recipients.

In block 1120, an outcome measurement may be computed for each of the plurality of patients at a second point in time. According to certain embodiments, the outcome measurement may represent a mortality of each of the patients. For example, the mortality may be determined by reviewing death records available at the National Institute of Standards and Technology. According to certain embodiments, the outcome measurements may be determined at a second time corresponding to ninety-days post discharge. According to certain embodiments, the outcome measurements may be determined at a second time corresponding to one-year post discharge. Generally, the patient 202 will not have been discharged until the patient 202 is stable enough to be discharged safely. The patient 202 may be discharged to home or to a skilled nursing facility. Unfortunately, in some instances, the patient 202 may die. According to certain embodiments, the outcome measurement is either a “yes” or “no” answer. For example, was this patient dead one-year post discharge?

In block 1130, a dataset may be generated where each of the patients has data from the first point in time and an outcome measurement from the second point in time. The dataset may be considered a set of (x,y) pairs for each patient, wherein x is the patent data at the first time, and wherein y is the outcome measurement at the second time.

In block 1140, the (x,y) pairs of the dataset may be ordered or sorted. In block 1150, the (x,y) pairs of the dataset may be binned to form a plurality of binned data sets. According to certain embodiments, the bin size for each of the binned data sets is selected to have at least two percent of the total number of (x,y) pairs. According to certain embodiments, the (x,y) pairs are binned based on the values of x, which represents the patient data from the first point in time.

In block 1160, an average value (x_bar) for the patient data from the first point in time (x) and an average value (y_bar) for the outcome measurements (y) may be computed. According to certain embodiments, the dataset has enough (x,y) pairs so that each bin size has a sufficient number of (x,y) pairs so that the average value for x (x_bar) and the average value for y (y_bar) are statistically significant. According to certain embodiments, the dataset has pairs (x,y) spanning the values of x that are of interest. Given that the majority of values of x will be close to the average value for x (x_bar), and given that the impact of deviations from the average are generally of great interest, the dataset may need to be large, on the order of thousands of pairs.

In block 1170, the minimum average value of outcome measurements (y_bar_min) may be subtracted from each outcome measurement (y) for each binned data set resulting in a new shifted dataset. Once y_bar_min is subtracted from each outcome measurement (y), a new value of y_bar_min may be shown to be zero.

In block 1180, a specific function y=f(x) may be derived for assessing excess risk associated with the patient data. According to certain embodiments, the function y=f(x) can define the excess risk due to deviation from normative values for the medical data. According to certain embodiments, if the type of medical data that is collected from an EMR is a continuous variable or an ordinal score, the specific function y=f(x) is a sum of a first function defined from average minimum value of x to the average maximum value of x, a second constant function that covers values of x less than x_bar_min, and a third constant function that covers values of x greater than x_bar_max. According to certain embodiments, if a value for x is greater then x_bar_max then the value of y is the value of y_bar that corresponds to the value of x bar_max, and if a value for x is less then x_bar_min then the value of y is the value of y_bar which corresponds to the value of x_bar_min. According to certain embodiments, the first function is derived by curve fitting through the (x_bar, shifted_y bar) points, to provide smooth interpolation between all of the x_bar values and all of the shifted y_bar values. According to certain embodiments, the curve may not used to extrapolate beyond the highest or lowest values of x_bar. According to certain embodiments, the first function is derived by fitting an appropriate functional form. According to certain embodiments, the functional form may be selected from the group consisting of a line, a parabola, a polynomial, a sine function, and an exponential function. According to certain embodiments, the first function may be derived by fitting a higher order polynomial derived using a linear least squares method. The curve can represent the first function defined from average minimum value of x to the average maximum value of x. According to certain embodiments, the curve that is fit through the points is well-behaved, that is, the curve smoothly interpolates between all of the x_bar values and all of the new y_bar values. According to certain embodiments, if the type of data that is collected is a categorical class or a binary assessment, the function y=f(x) may be if x=“a” then y=y_bar value for just value “a.” According to certain embodiments, the function may be used in computing a health score for a patient 202. According to certain embodiments, the function may be used when lab results (or similar patient data) are reported to a recipient. The function may give the recipient a sense of the implications from a particular value of the medical data. According to certain embodiments, the function may be used to help researchers better understand physiology.

According to certain embodiments, it may be desirable to create a two-dimensional array from the binned data sets by associating the x_bar value for each binned data set with one axis of the two-dimensional array and associating the corresponding new y_bar value for each binned data set with a second axis of the two-dimensional array.

According to certain embodiments, the type of medical data that is collected may be a continuous variable. Examples of continuous variables may include, but are not limited to, medical data obtained from a blood chemistry panel screen, medical data relating to an arterial blood gas (ABG) test, medical data relating to a blood analysis test, and medical data measuring a vital sign value. In an embodiment, the blood chemistry panel screen includes medical data relating to an albumin/globulin (A/G) ratio, an alanine aminotransferase (ALT or SGPT) value, an aspartate aminotransferase (AST or SGOT) value, an albumin value, an alkaline phosphatase value, a blood urea nitrogen (BUN) value, a calcium value, a carbon dioxide (CO2) value, a chloride value, a creatinine value, a globulin value, a glucose value, a potassium value, a sodium value, a total bilirubin value, a total protein value and a troponin value. In an embodiment, the arterial blood gas test includes medical data relating to a base excess value, a fraction of inspired oxygen (FiO2) value, a bicarbonate (HCO3) value, a partial pressure of carbon dioxide (PCO2) value, a partial pressure of oxygen (PO2) value and a pH value. In an embodiment, the blood analysis test includes medical data relating to a hematocrit percentage, a hemoglobin value and a white blood cell count. In an embodiment, the vital sign value includes medical data relating to a heart rate value, a diastolic blood pressure value, a systolic blood pressure value, a respiration rate, a percentage of arterial hemoglobin in the oxyhemoglobin configuration (pulse Ox) and a temperature value. According to certain embodiments, the type of medical data that is collected may be an ordinal score, such as a Braden scale score.

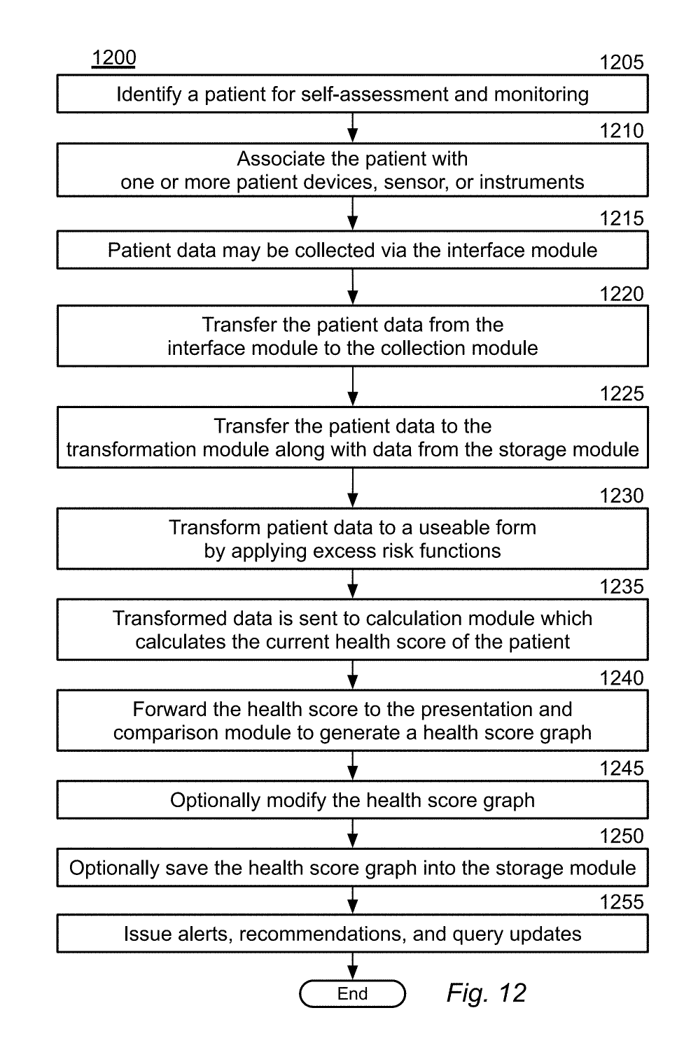

FIG. 12 is a block flow diagram depicting a method 1200 for determining an overall risk associated with the current health condition of a patient 202, in accordance with certain exemplary embodiments. In block 1205, a patient 202 is identified for self-assessment and monitoring. At block 1210, the patient 202 can be associated with one or more wearable sensor devices 100, sensors, or instruments for collecting patient data.

At block 1215, the interface module 210 may begin obtaining the patient data and importing the patient data into the health scoring system 208. Some patient data may be obtained directly from the patient 202 via patient queries. Other patient data may be obtained from sensors, or medical instruments. Still other patient data may be obtained from electronic medical records or caregiver reports. The patient data may include any number of the medical statistics or subjective valuations that may be used to generate the health score.

At block 1220, the data may be transferred along to the collection module 220. The collection module 220 may be coupled to the interface module 210 for receiving the various patient data. The collection module 220 may store the patient data into the storage module 290.

At block 1225, the patient data may be transferred to the transformation module 240. Additionally, past patent data 210 (for example, previous health scores of the patient 202) may also be collected and transferred to the transformation module 240.

According to certain embodiments, generation of one or more health scores may include both subjective and objective data. Subjective data, such as self-assessments obtained through patient query responses, may be significant in predicting the health of the patient 202. Traditionally, clinical caregivers often overlook this information, yet its inclusion can increase the robustness of the health score, and may provide a channel for patients 202 to more effectively contribute to their care.

According to certain embodiments, a single term in the health score formula may contain multiple medical data inputs. For example, various pieces of patient data (e.g. blood pressure, heart rate, and similar readings) may each be transformed into a particular number, which are then combined to form an over all health score. It should be understood, however, the multiple patient data inputs may be combined before being transformed, such that the transformed number used for forming a portion of the health score, may be a combination of multiple health readings. For example, systolic and diastolic blood pressure may be combined into a single number before being transformed for use in the health score. The collection module 220 may obtain both past and present data necessary for the patient 202 on each of the categories to form one or more health scores.

Next, at block 1230, the patient data may be transformed into a usable format. The transformation module 240 may carry out the transformation such that all of the disparate forms of patent data may be readily combined with one another. The transformation module 240 may be configured to transform each of the pieces of patient data obtained from collection module 220 into a numerical quantity. The transformation performed by the transformation module 240 may include any number of mathematical or logical operations. Transformations may also take multiple inputs to produce a single transformed output. Multiple inputs may include historical data for the patient 202 or for any given class of patients. The transformation module 240, after receiving patent data from the collection module 220, may process the data and transform it into values for use in generating one or more health scores for the patient 202. The transformation module 240 may convert each piece of patient data to health score values using a set of functions stored within the health score system 208. These stored functions may define excess risk due to deviation from normative values for each of the patient data.

At block 1235, the transformed patient data may combined into one or more health scores. The transformed patient data may be transferred to the combination module 250 for combining into a health score according to a predetermined algorithm. The combination module 250 may be configured to take the transformed quantities from transformation module 240, apply weighting modifiers, combine them, and then scale them onto a range, such as a score between 0 and 100. The health score, generated by combination module 250, may be based on the various health factors measured and transformed above, the resulting heal score may thus be a relative overall health score of the patient 202 being monitored.

According to certain embodiments, weighting factors (two times, three times, and more) can be added or multiplied to certain transformed numbers as applicable to specific patient conditions. For example, respiratory factors may be weighted more heavily when a particular patient 202 is recovering from a lung-based ailment such as pneumonia. Likewise, similar weighting factors can be applied to the transformed scores of heart rate, heart rhythm, systolic and diastolic pressure for patients with heart conditions. It is understood that any number of modifications introduced into a similar combination module 250, or within the health score system 208 in general, is within the spirit and scope of the technology presented herein.

At block 1240, the health score may be transmitted to the presentation and/or comparison module 260. The presentation and/or comparison module 260 may use the current health score, as well as historical data from storage module 290 (past health scores), to generate a health score graph.

The presentation and/or comparison module 260 of health score system 208 may be configured to import the various data components compiled by combination module 250. This data may be used to render a health score graph for the patient 202. According to certain embodiments, the presentation and/or comparison module 260 may also render a statistical reference curve on the health score graph. This may support, for example, the present health score being easily compared to an average patient with similar conditions and circumstances. According to certain embodiments, the presentation and/or comparison module 260 may supply principal corresponding measurements of direct patient data onto the health score graph. A smoothed health score curve may also be provided alongside the health score graph to provide a running average of the health score over time. The curvature of a smoothed health score graph may also be provided.

Statistical reference curves may also be added to health score graph. For example, when such information is available, statistically computed average patient health score trajectories, for each specific procedure and initial patient condition, may be included on chart next to the health score plot. This information may be stored in the storage module 290, and may be imported into the comparison module 260 by the collection module 220. Statistical reference curves may include linear information with standard deviation error bars or transformed values. If the patient 202 is below expectation by a certain number of standard deviations, the system may generate an alert using alert module 270.

Further granularity may be provided with respect to the statistical reference curves. For example, instead of having a single reference curve for average open-heart patients of age 80, it can be further broken down by gender, and even further modified as to a patient's initial condition by using only patients with similar health scores at the time of discharge from the hospital.

Principal corresponding measurement curves may also be generated. The health score graph may provide an instant context and patient health trajectory. It may also be important for recipients to have access to other direct measurements, including, but not limited to, diastolic blood pressure, temperature, respiration rate, pulse, and pain score. This can support the detection of other trends that may be affecting the health score and, thus, the patient 202. It should be understood that, when using the option of adding direct patient data to the health score graph, the health score system 208 has the ability to let the healthcare provider select which principal corresponding measurements they would like to see. When the health score is improving or is adequate, such features may be toggled off, as they are less important in such instances.

According to certain embodiments, the presentation and/or comparison module 260 may be configured to alter the health score graph so that when a healthcare provider detects a trend in the health score plot, they can understand exactly what factors are contributing. Accordingly, the heal score system 208 may provide for a component expansion window, such that if the patient has a health score 145 of 65 (for example), the expansion might show that the patient lost 12 points due to elevated temperature (over 101 Fahrenheit), lost 18 points due to rapid pulse (between 100 and 202 beats per minute) and lost 5 points due to a self-assessment pain score of five; all out of the perfect health score of 100.

According to certain embodiments, the presentation and/or comparison module 260 may also alter the health score graph to obtain certain kinds of slope information. Even though trends may be easy to spot by eye upon looking at health score plot, an automatic “simple” slope calculation may also be useful. Mathematically, this is the first derivative of the health score as a function of time. Due to the “noisiness” of typical health score plots some averaging methods may be employed as well. If the slope is positive, the patient is probably getting better; if it is approximately zero, then the patient is staying the same; and if it is negative, then the patient may be getting worse. Slope lines may be added to the health score plot Such slope information may help identify trends in health score plot, particularly, when the plot is “noisy” due to large variations between each health score measurement.

The presentation and/or comparison module 260 of the health score system 208 may also compute “rate of change” of the simple slope. For instance, although the patient 202 may be improving, the rate of improvement may be decreasing. Such a slowing of recovery could be evidence of a problem just beginning to develop. Mathematically, this curvature information is the second derivative of the health score as a function of time. Similar to the slope data, due to the “noisiness” of the curves, averaging (or smoothing) may be included in the computation.

When the patient data or the health score is noisy, a “running average” or other “smoothing” of the health score can be displayed on health score graphs. The smoothed health score curve could incorporate both the first derivative (slope) and/or the second derivative (curvature) by color-coding or by thickness of the displayed line. For example, if the patient 202 was getting worse (negative slope), the line might be colored red. As other examples, if the patient is getting worse at an accelerating rate, or is getting better at a lessening rate, then the line may be bolded for emphasis.

It should be appreciated that such modifications to patient health score graphs are intended only as example modification and are in no way intended to limit the scope of the present disclosure. Any similar disclosure that utilizes modified health score graphs is also within the spirit and scope of the technology presented herein.

At block 1245, the health score graph may be modified as discussed herein. At block 1250, the health score or the health score graph may be saved. For example, they may be save to the storage module 290. At block 1255, the alert module 270 may generate an alert for delivery to one or more recipient as discussed herein.

Example Systems

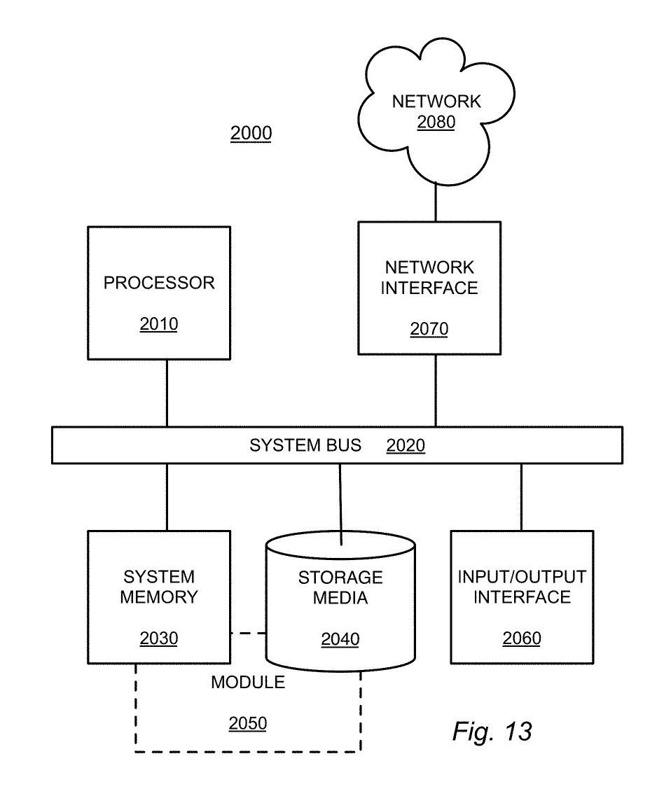

FIG. 13 depicts a computing machine 2000 and a module 2050 in accordance with one or more embodiments presented herein. The computing machine 2000 may correspond to any of the various computers, servers, mobile devices, embedded systems, or computing systems presented herein. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a vehicular information system, one more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, co-processors, or any combination thereof. According to certain embodiments, the processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 also may include volatile memories, such as random access memory (“RAM”), static random access memory (“SRAM”), dynamic random access memory (“DRAM”), and synchronous dynamic random access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid sate drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for operably coupling the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCP”), PCI express (PCIe), serial bus, parallel bus, advanced technology attachment (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, biometric readers, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include wide area networks (“WAN”), local area networks (“LAN”), intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals discussed herein through the system bus 2020. It should be appreciated that the system bus 2020 may be within the processor 2010, outside the processor 2010, or both. According to some embodiments, any of the processor 2010, the other elements of the computing machine 2000, or the various peripherals discussed herein may be integrated into a single device such as a system on chip (“SOC”), system on package (“SOP”), or ASIC device.

In situations in which the systems discussed here collect personal information about users, or may make use of personal information, the users may be provided with a opportunity to control whether programs or features collect user information (e.g., information about a user's social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

One or more aspects of embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the invention should not be construed as limited to any one set of computer program instructions. Further, a skilled programmer would be able to write such a computer program to implement an embodiment of the disclosed invention based on the appended flow charts and associated description in the application text. Therefore, disclosure of a particular set of program code instructions is not considered necessary for an adequate understanding of how to make and use the invention. Further, those skilled in the art will appreciate that one or more aspects of the invention described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (“FPGA”), etc.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of embodiments of the invention. Accordingly, such alternative embodiments are included in the inventions described herein.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of the technology presented herein which is to be accorded the broadest interpretation so as to encompass such modifications and equivalent structures.

Although the subject matter presented herein has been described in specific language related to structural features or methodological acts, it is to be understood that the invention defined in the appended claims is not necessarily limited to the specific features or acts described herein. Rather, the specific features and acts are disclosed as example forms of implementation.

The subject matter described above is provided by way of illustration only and should not be construed as limiting. Various modifications and changes may be made to the subject matter described herein without following the example embodiments and applications illustrated and described, and without departing from the true spirit and scope of the present invention, which is set forth in the following claims.

What is claimed is:

1. A method for adaptive health score tracking comprising:
registering a patient;
establishing communications with a wearable sensor device associated with the patient;
receiving physiological sensor data associated with patient from the wearable sensor device;
calibrating the wearable sensor device according to the received physiological sensor data;
generating athletic performance benchmarks associated with the patient according to the received physiological sensor data;
applying data analytic processes to the physiological sensor data and the athletic performance benchmarks;
computing a periodic health score in response to results from the data analytic processes; and
transmitting actionable information for athletic training in response to the periodic health score.

2. The method of claim 1, further comprising transmitting actionable information for athletic strategy in response to the periodic health score.

3. The method of claim 1, wherein the patient is a human athlete.

4. The method of claim 1, wherein the patient is a non-human animal.

5. The method of claim 1, wherein physiological sensor data associated with patient comprises one of heart rate, respiratory rate, blood pressure, and movement.

6. The method of claim 1, wherein calibrating the wearable sensor device comprises comparing the physiological sensor data from the wearable sensor device with stored corresponding measurements from a population of other patients.

7. The method of claim 1, wherein changes in the periodic health score signal deterioration in performance correlated to changes in the physiological sensory data from the wearable sensor device.

8. The method of claim 1, wherein the actionable information for athletic training comprises timing of training sessions and recovery periods.

9. The method of claim 1, wherein the actionable information for athletic training comprises dietary intake.

10. The method of claim 1, wherein the actionable information comprises types of medications and dosage of medications.

11. A system for adaptive health score tracking comprising:
a housing operable to be worn on the body of a patient;
a plurality of physiological sensors operable to measure a heart rate, a respiratory rate, a blood pressure, and motion metrics associated with the patient;
a bidirectional radio frequency data interface; and
one or more processing units associated with one or more processing modules configuring the one or more processing units to:
establish communications, via the bidirectional radio frequency data interface, with a cloud-based computing platform,
register the patient with the cloud-based computing platform,
receive physiological sensor data from the plurality of physiological sensors,
calibrate the received physiological sensor data in conjunction with the cloud-based computing platform,
generate athletic performance benchmarks associated with the patient according to the received physiological sensor data,
apply data analytic processes to the physiological sensor data and the athletic performance benchmarks,
compute a periodic health score in response to results from the data analytic processes, and
transmit actionable information for athletic training in response to the periodic health score.

12. The system of claim 11, wherein the housing comprises a wristband.

13. The system of claim 11, wherein the housing comprises a remote patch.

14. The system of claim 11, wherein calibrating comprises comparing the physiological sensor data with corresponding measurements made in a clinical or laboratory setting.

15. The system of claim 11, wherein calibrating comprises comparing the physiological sensor data with corresponding measurements from a population of other patients stored within the cloud-based computing platform.

16. The system of claim 11, wherein changes in the periodic health score signal deterioration in performance correlated to changes in the physiological sensory data from the wearable sensor device.

17. The system of claim 11, wherein computing the periodic health score comprises applying an excess risk model.

18. The system of claim 11, wherein the actionable information for athletic training comprises timing of training sessions and recovery periods.

19. The system of claim 11, wherein the actionable information for athletic training comprises dietary intake.

20. The system of claim 11, wherein the actionable information comprises types of medications and dosage of medications.
